SEQUENCE LISTING (26 sequences)

SEQ ID NO: 1 - DNA, 18 bp, Artificial Sequence
  5′ external gDNA probe
ggaagtatgacatcaaag

SEQ ID NO: 2 - DNA, 20 bp, Artificial Sequence
  5′ external gDNA probe
tagaggttgtggggaagata

SEQ ID NO: 3 - DNA, 24 bp, Artificial Sequence
  internal gDNA probe
gtcatgggggctgctgtgtttttc

SEQ ID NO: 4 - DNA, 23 bp, Artificial Sequence
  internal gDNA probe
gaaggacagagacagcagcacca

SEQ ID NO: 5 - DNA, 23 bp, Artificial Sequence
  3′ external gDNA probe
agtccatactggccctgtattca

SEQ ID NO: 6 - DNA, 24 bp, Artificial Sequence
  3′ external gDNA probe
aggcattagaatcagctcagagca

SEQ ID NO: 7 - DNA, 20 bp, Artificial Sequence
  5′ external gDNA probe
ctgaagcctgggatgaagtt

SEQ ID NO: 8 - DNA, 20 bp, Artificial Sequence
  5′ external gDNA probe
tgtgacgtggccagtgtatt

SEQ ID NO: 9 - DNA, 25 bp, Artificial Sequence
  3′ external gDNA probe
atgcgactgttggcctatggtaaag

SEQ ID NO: 10 - DNA, 20 bp, Artificial Sequence
  3′ external gDNA probe
catatgcgtgtgtgtgtatg

SEQ ID NO: 11 - DNA, 23 bp, Artificial Sequence
  5′ external gDNA probe
cttgctgtggagcagctcgagga

SEQ ID NO: 12 - DNA, 24 bp, Artificial Sequence
  5′ external gDNA probe
agtggatccgaggtgactgggacg

SEQ ID NO: 13 - DNA, 25 bp, Artificial Sequence
  internal cDNA probe
cttctggttggtgtctctcctgctt

SEQ ID NO: 14 - DNA, 24 bp, Artificial Sequence
  internal cDNA probe
ggagaatcaccatgaatgcccact

SEQ ID NO: 15 - DNA, 25 bp, Artificial Sequence
  3′ external gDNA probe
gctcttggctaatgcctgaagaaga

SEQ ID NO: 16 - DNA, 22 bp, Artificial Sequence
  3′ external gDNA probe
ggataacacagggttgcaacca

SEQ ID NO: 17 - DNA, 24 bp, Artificial Sequence
  oligonucleotide primer
tatccagcgcagcctttcgtgccg

SEQ ID NO: 18 - DNA, 21 bp, Artificial Sequence
  oligonucleotide primer
cccccatgttccctcagtccc

SEQ ID NO: 19 - DNA, 24 bp, Artificial Sequence
  oligonucleotide primer
tatccagcgcagcctttcgtgtaa

SEQ ID NO: 20 - DNA, 21 bp, Artificial Sequence
  oligonucleotide primer
cagcgaggagacggaggatga

SEQ ID NO: 21 - DNA, 21 bp, Artificial Sequence
  oligonucleotide primer
atcacccatctccatccgaca

SEQ ID NO: 22 - DNA, 23 bp, Artificial Sequence
  oligonucleotide primer
gcccaagtgcatcagccaaaata

SEQ ID NO: 23 - DNA, 24 bp, Artificial Sequence
  oligonucleotide primer
tccgctaagaaatcgtcccagtca

SEQ ID NO: 24 - DNA, 21 bp, Artificial Sequence
  oligonucleotide primer
cgtgaggagggtgtaccactt

SEQ ID NO: 25 - DNA, 19 bp, Artificial Sequence
  oligonucleotide primer
gactggctcccgaggtcgt

SEQ ID NO: 26 - DNA, 18 bp, Artificial Sequence
  oligonucleotide primer
aggagagacaccaaccag

(19) United States
(12) Patent Application Publication
(10) Pub. No.: US 2008/0120731 A1
(43) Pub. Date: May 22, 2008

(54) Transgenic Animal Models for Neurodevelopmental Disorders

(76) Inventors: Bart De Strooper, Leuven (BE); Tim Dejaegere, Leuven (BE); Ludgarde Serneels, Heist Op Den Berg (BE)

Correspondence Address: NIXON & VANDERHYE, PC, 901 NORTH GLEBE ROAD, 11TH FLOOR, ARLINGTON, VA, 22203, US

(21) Appl. No.: 11/792,380

(22) PCT Filed: Dec. 13, 2005
(86) PCT No.: PCT/EP05/56753

§ 371 Date: Jun. 6, 2007

Related U.S. Application Data
(60) Provisional application No. 60/635,182, filed on Dec. 13, 2004.
(60) Provisional application No. 60/681,476, filed on May 17, 2005.

(30) Foreign Application Priority Data
Dec. 13, 2004 (EP) 04106516.0
May 17, 2005 (EP) 05104110.1

(51) Int. Cl. C12Q 1/34; A01K 67/027; C12N 5/06; C12N 9/48; G01N 33/50

(52) U.S. Cl. 800/3; 800/13; 800/18; 800/9; 435/325; 435/212; 435/18

(57) ABSTRACT

The current invention relates to the field of neurodevelopmental disorders and more particularly to the field of neuropsychiatric disorders. The invention provides non-human, transgenic animal models for said neurodevelopmental disorders such as schizophrenia, bipolar disorders, compulsive disorders and the like. The animals also have applications in the field of Alzheimer's Disease and other disorders in which γ-secretase activity has a role.

### FIELD OF THE INVENTION

The current invention relates to the field of neurodevelopmental disorders and more particularly to the field of neuropsychiatric disorders. The invention provides non-human, transgenic animal models for said neurodevelopmental disorders such as schizophrenia, bipolar disorders, compulsive disorders and the like. The animals also have applications in the field of Alzheimer's Disease and other disorders in which γ-secretase activity has a role.

### INTRODUCTION TO THE INVENTION

γ-Secretase is the proteolytic activity responsible for the cleavage of a series of integral membrane proteins, most notoriously the Amyloid Precursor Protein (APP) and Notch. Generally γ-secretase cleaves the hydrophobic integral membrane domain of its substrates (except for N-cadherin), resulting in the release of protein fragments at the luminal (extracellular) and at the cytoplasmic side of the membrane (Annaert and De Strooper, 2002). In the case of Notch and some other substrates, the released cytoplasmic domains interact with DNA binding proteins and regulate gene transcription, linking γ-secretase function to a series of signalling processes. The catalytic part of the protease is contributed by the presenilin protein (De Strooper et al., 1998; Li et al., 2000; Wolfe et al., 1999). Mutations in the presenilin gene are the cause of a familial form of Alzheimer's Disease (Sherrington et al., 1995). The Presenilins (PSEN) appear to provide the active core of the protease. Two mammalian homologues, PSEN1 and PSEN2, exist. The PSEN (˜50 kDa) span the cellular membranes several times. Two aspartate residues (Asp 257 and Asp 385) located in transmembrane domains 6 and 7 respectively, are essential for the catalytic activity of the protease. Although the working mechanism needs further scrutiny, γ-secretase may therefore indeed be considered an aspartyl protease (Wolfe et al., 1999). PSEN are synthesized as precursor proteins that must become incorporated into a larger complex for stabilization. The pool that is not incorporated into these complexes is rapidly degraded by the proteasome. The stabilization of PSEN is accompanied by a proteolytic “maturation” cleavage performed by an unknown “presenilinase” (Thinakaran et al., 1996). The resulting amino-terminal fragment (NTF ˜30 kDa) and carboxy-terminal fragment (CTF ˜20 kDa) contribute each separately one aspartyl residue to the catalytic site. Both fragments are part of a larger complex. The exact molecular weight of this complex is an issue of debate and varies according to the techniques used. The minimal estimate is 200-250 kDa but ˜440 kDa (Edbauer et al., 2002) and even larger complexes have been described. Using antibodies against the PSEN fragments, a second member of the complex, called Nicastrin (Nct), was purified (Yu et al., 2000). Nct is a glycosylated ˜130 kDa integral membrane protein that binds relatively well to both the NTF and the CTF of PSEN. Goutte and colleagues used a screen for genes that cause an “anterior pharynx defective phenotype” reflecting deficient glp1 signalling (glp1 and lin12 are the two Notch receptors in C. elegans). They identified two such genes called Aph1 and Aph2. Aph2 is the homologue of mammalian Nct. Aph1 is a novel ˜30 kDa multi-membrane spanning protein that, similar to Psen, is needed for the correct subcellular transport of Aph2/Nct to the cell surface (Goutte et al., 2002). Aph1 (Pen1) was also identified independently in a screen for Presenilin enhancers that cause a glp-1 sterility in a C. elegans strain partially deficient in Psen (Francis et al., 2002). This screen yielded, in addition, the fourth γ-secretase partner: Pen2. Pen2 is a small, hairpin like membrane protein with Mr ˜12 kDa. Francis et al. (2002) demonstrated that Aph1 and Pen2 act at, or upstream, of the release of the Notch intracellular domain, like Presenilin does. Down-regulation of one of the two new proteins in cell culture via siRNA leads to a decline in γ-secretase activity (Lee et al., 2002), comparable to what was demonstrated before with Nct (Edbauer et al., 2002) and Presenilin (De Strooper et al., 1998). Thus, all four proteins are needed for cleavage of Notch and APP substrates. Over-expression of any combination of three proteins does not increase processing of APP. Over-expressing the four proteins together results concomitantly in the processing and stabilization of Psen, the increased expression of fully glycosylated Nct, and a clear enhancement of γ-secretase activity in cell based and cell free assays. Thus it seems that the minimal number of components needed for the proteolytic activity of the complex have been identified, Pen2 and Aph1 being apparently the long sought “limiting cellular factors” controlling Psen expression (Thinakaran et al., 1996). In mammalian species several paralogues of the individual prototype proteins and a series of alternative spliced forms of Aph1A have been identified. From the loss of function and over-expression experiments performed in different species it is observed that the four basic components of the γ-secretase activity influence each other's stability and maturation. The available evidence shows that the four proteins are subunits of a larger, relatively stable active complex. As already mentioned, γ-secretase cleaves quite a broad range of substrates with a relaxed specificity. In fact, γ-secretase cleaves almost by default any type I integral membrane protein whose ectodomain is shorter than a certain number of amino acid residues (Struhl and Adachi, 2000). If the total molecular weight of the individual subunits is taken together, a close approximation of the estimate for the minimal molecular weight of the intact complex, i.e. 200-250 kDa, is obtained. This implies a 1:1:1:1 stoichiometry. Therefore, taking into account the two mammalian Psen and the two (or three in rodents) Aph1 homologues, the existence of at least four different γ-secretase complexes in mammalian species, can be inferred. Moreover in rodent a gene duplication event has given raise to a third Aph1C gene. In the present invention we have constructed a series of Aph1 deficient mice. Surprisingly these mice are altered in behavioural and pathological aspects that reflect human neurodevelopmental disorders like schizophrenia, bipolar disorder and severe depression, autism, attention deficit hyperactivity disorder (ADHD), mental retardation, and others. These transgenic mice are valuable models for studying symptoms related to one or more neurodevelopmental disorders. These mice and cell lines derived thereof can further be used for testing compounds having therapeutical effects with respect to these diseases and Alzheimer's Disease

### FIGURES

FIG. 1. Targeted disruption of the Aph1 genes by homologous recombination.

Maps of the targeting vectors, the wild-type Aph1 alleles, the conditional targeted alleles (floxed allele), and the disrupted Aph1 alleles from Aph1A (A) Aph1B and Aph1C (B) respectively are shown. A schematic drawing of chromosome 9 showing the clustered Aph1C and Aph1B genes is shown. Exons are indicated as black boxes. LoxP and FRT (FLP mediated recombination can remove the selection marker cassette) recombination sites are indicated as black arrowhead and white flags respectively. Arrows indicate the locations of PCR primers. The expected sizes for the indicated restriction enzyme digested fragments detected by 5′(L), 3′(R) flanking or internal probes (PCR fragments, black bars) from targeted and wild-type alleles are indicated below every construct with line diagrams. Positive selection marker genes and reporter genes are indicated as colored boxes. The box marked LACZ represents an engineered LacZ reporter gene (3′ splice acceptor site and polyadenylation signal). The box marked hu-ALPP represents an engineered AP reporter gene (polyadenylation signal included). Relevant restriction sites are shown Sph (SpHI), EV (EcoRV), Stu (Stul), Spe (Spel).

FIG. 2. Analysis of APP processing in the brain (A): Western blot analysis of brain extracts from wt mice (wt) and Aph1BC^{-/-}littermate mice using antibodies against APP (CTF), Psen-1 (NTF), Nct, Pen-2 and actin as a loading control. (B) Quantification of the relative accumulation of APP-CTFs. The densitometric values obtained for APP-CTF in Aph1BC^{-/-} brain regions were normalized to the average signal for APP-CTF in the corresponding wild-type region (=100%). Statistically significant differences are indicated by asterisks (*: p<0,05; ***: p<0,001). The number of independent mice analysed per brain region is indicated at the bottom of each graph.

FIG. 3. For all trials, background noise was 70 db, the prepulse preceded the startle stimulus by 100 ms, the prepulse stimuli lasted 20 ms and the startle stimuli lasted 60 ms. All stimuli consisted of white noise. The interval between the trials varied between 10 and 15 s. For each of the four different combinations of prepulse and startle stimulus, the % PPI was calculated. Compared to wild-type littermates, APH1BC-deficient mice showed a highly significantly reduced PPI for 110 db trials (p<0,001 for genotype effect in a 2-way repeated measures ANOVA with genotype and trial type as factors). For both prepulse 74/pulse 110 and prepulse 78/pulse 110 trial types, PPI in the knockouts was 70-75% of wild-type levels (post-hoc comparisons: p=0,001 for prepulse 74/pulse 110, and p=0,002 for prepulse 78/pulse 110 trials). For 100 db trial types, there was also a PPI-impairment in the knock-outs, but it was less outspoken and only moderately significant (p=0,029 for genotype effect). Post-hoc comparisons revealed that the impairment was only significant for prepulse 74/pulse 100 trial types (p=0.011).

### DETAILED DESCRIPTION OF THE INVENTION

The present invention discloses transgenic animals that are suitable animal model systems to study neurodevelopmental disorders. Said neurodevelopmental disorders are complex neuropsychiatric disorders comprising schizophrenia, bipolar disorder, severe depression, autism, attention deficit hyperactivity disorder (ADHD), lissencephaly and mental retardation. The transgenic animals are engineered such that they lack expression of the Aph1a and/or the Aph1b and/or the Aph1c gene in at least one tissue or organ. The transgenic animals of the present invention display symptoms that are relevant for one or more neurodevelopmental disorders. In other words, display symptoms, which are shared by one or more neurodevelopmental disorders. Further, the transgenic animals provide a test system for the evaluation of strategies for diagnosis, prevention or therapeutic intervention. In addition, the animals may also be utilized in toxicological investigations designed to identify and evaluate environmental factors that contribute to the development of neurodevelopmental disorders. They can finally be used to explore the differential distribution of different γ-secretase complexes to the overall γ-secretase activity and to screen for inhibitors specific or more specific for one of the different γ-secretase complexes (i.e. PS1/APH1A or PS1/APH1B-C or PS2/APH1A or PS2/APH1B-C containing complexes, Nct and Pen-2 supposed to be constant).

The term “neurodevelopmental disorder” refers to a specific medical disease or condition that causes a developmental disability due to a dysfunction/disease of the central nervous system. Consequently a neurodevelopmental disorder can be either “genetic” or “acquired”. Regardless of the exact cause, most people with neurodevelopmental disorders will have one or more of four “general” complications, namely: cognitive disability, neuromotor dysfunction, seizures, or abnormal behaviours. The term “animal” is used herein to include all vertebrate animals, except humans. It also includes an individual animal in all stages of development, including embryonic and foetal stages. A “transgenic animal” is any animal containing one or more cells bearing genetic information altered or received, directly or indirectly, by deliberate genetic manipulation at the subcellular level, such as by targeted recombination or microinjection or infection with recombinant vector. The term “transgenic animal” is not meant to encompass classical cross-breeding or in vitro fertilization, but rather is meant to encompass animals in which one or more cells are altered by or receive a recombinant DNA molecule as described above. The latter molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring in fact, possess some or all of that alteration or genetic information, then they, too, are transgenic animals. The alteration or genetic information may be foreign to the species of animal to which the recipient belongs, or foreign only to the particular individual recipient, or may be genetic information already possessed by the recipient. In the last case, the altered or introduced gene may be expressed differently than the native gene (e.g. lack of expression in a specific organ or tissue).

In a first embodiment the invention provides a transgenic, non-human animal characterised by having an endogenous nucleic acid sequence encoding a non-functional aph1A and/or aph1B and/or aph1C expression. In another embodiment the invention provides a transgenic, non-human animal characterised by having an endogenous nucleic acid sequence encoding a non-functional aph1B. In another embodiment the invention provides a transgenic, non-human animal characterised by having an endogenous nucleic acid sequence encoding a non-functional aph1C. In another embodiment the invention provides a transgenic, non-human animal characterised by having an endogenous nucleic acid sequence encoding a non-functional aph1B and aph1C. A transgenic, non-human animal characterised by having an endogenous nucleic acid sequence encoding a non-functional aph1B and aph1C is considered as a model for total aph1B loss in humans. Indeed, in humans aph1C does not exist. In rodents Aph1B and C are highly similar (96.3% at the nucleotide level) and both genes are clustered on chromosome 9. Most likely they arose by rodent-specific gene duplication. In yet another embodiment the invention provides a transgenic, non-human animal characterised by having an endogenous nucleic acid sequence encoding a non-functional aph1A and/or aph1B and/or aph1C expression wherein said non-functional aph1A and/or aph1B and/or aph1C expression is in a specific tissue or in a specific organ.

Thus in other words the present invention provides a transgenic non-human animal in which in at least one organ or tissue the Aph1A and/or Aph1B and/or Aph1C gene has been selectively inactivated. In a preferred embodiment the non-functional expression of the Aph1A and/or Aph1B and/or Aph1C gene is in the brain or in a specific region of the brain. More specifically, the present invention provides a transgenic non-human animal whose genome comprises a disruption in an Aph1A and/or Aph1B and/or Aph1C gene, wherein the transgenic animal exhibits a decreased level of functional Aph1A and/or Aph1B and/or Aph1C protein relative to wild-type. The non-human animal may be any suitable animal (e.g., cat, cattle, dog, horse, goat, rodent, and sheep), but is preferably a rodent. More preferably, the non-human animal is a rat or a mouse. Unless otherwise indicated, the term “Aph1A and/or Aph1B and/or Aph1C gene” refers herein to a nucleic acid sequence encoding Aph1A and/or Aph1B and/or Aph1C protein, and any allelic variants thereof. Due to the degeneracy of the genetic code, the Aph1A and/or Aph1B and/or Aph1C gene of the present invention include a multitude of nucleic acid substitutions which will also encode an Aph1A and/or Aph1B and/or Aph1C protein. An “endogenous” Aph1A and/or Aph1B and/or Aph1C gene is one that originates or arises naturally, from within an organism. Additionally, as used herein, “Aph1A and/or Aph1B and/or Aph1C protein” includes both an “Aph1A and/or Aph1B and/or Aph1C protein” and an “Aph1A and/or Aph1B and/or Aph1C protein analogue”. A “Aph1A and/or Aph1B and/or Aph1C analogue” is a functional variant of the “Aph1A and/or Aph1B and/or Aph1C protein”, having an Aph1A and/or Aph1B and/or Aph1C-protein biological activity, that has 60% or greater (preferably, 70% or greater) amino-acid-sequence homology with the an Aph1A and/or Aph1B and/or Aph1C protein, as well as a fragment of the an Aph1A and/or Aph1B and/or Aph1C protein having an Aph1A and/or Aph1B and/or Aph1C-protein biological activity. As further used herein, the term “Aph1A and/or Aph1B and/or Aph1C-protein biological activity” refers to protein activity, which regulates gamma-secretase activity. Gamma-secretase activity can measured as described in (Nyabi et al, 2003). In yet another embodiment the invention provides cell lines derived from the above described transgenic animals, in particular cell lines lacking Aph1A, lacking Aph1B, lacking Aph1C and cell lines lacking Aph1B and C. In a particular embodiment said cells are primary neurons. As further used herein, the term “transgene” refers to a nucleic acid (e.g., DNA or a gene) that has been introduced into the genome of an animal by experimental manipulation, wherein the introduced gene is not endogenous to the animal, or is a modified or mutated form of a gene that is endogenous to the animal. The modified or mutated form of an endogenous gene may be produced through human intervention (e.g., by introduction of a point mutation, introduction of a frameshift mutation, deletion of a portion or fragment of the endogenous gene, insertion of a selectable marker gene, insertion of a termination codon, insertion of recombination sites, etc.). A transgenic non-human animal may be produced by several methods involving human intervention, including, without limitation, introduction of a transgene into an embryonic stem cell, newly fertilized egg, or early embryo of a non-human animal; integration of a transgene into a chromosome of the somatic and/or germ cells of a non-human animal; and any of the methods described herein.

The transgenic animal of the present invention has a genome in which the Aph1A and/or Aph1B and/or Aph1C gene has been selectively inactivated, resulting in a disruption in its endogenous Aph1A and/or Aph1B and/or Aph1C gene in at least one tissue or organ. As used herein, a “disruption” refers to a mutation (i.e., a permanent, transmissible change in genetic material) in the Aph1A and/or Aph1B and/or Aph1C gene that prevents normal expression of functional Aph1A and/or Aph1B and/or Aph1C protein (e.g., it results in expression of a mutant Aph1A and/or Aph1B and/or Aph1C protein; it prevents expression of a normal amount of Aph1A and/or Aph1B and/or Aph1C protein; or it prevents expression of Aph1A and/or Aph1Band/or Aph1C protein). Examples of a disruption include, without limitation, a point mutation, introduction of a frameshift mutation, deletion of a portion or fragment of the endogenous gene, insertion of a selectable marker gene, and insertion of a termination codon. As used herein, the term “mutant” is used herein to refer to a gene (or its gene product), which exhibits at least one modification in its sequence (or its functional properties) as compared with the wild-type gene (or its gene product). In contrast, the term “wild-type” refers to the characteristic genotype (or phenotype) for a particular gene (or its gene product), as found most frequently in its natural source (e.g., in a natural population). A wild-type animal, for example, expresses functional Aph1A and Aph1B and Aph1C.

Selective inactivation of a gene in a transgenic non-human animal may be achieved by a variety of methods, and may result in either a heterozygous disruption (wherein one Aph1A and/or Aph1B and/or Aph1C gene allele is disrupted, such that the resulting transgenic animal is heterozygous for the mutation) or a homozygous disruption (wherein both Aph1A and/or Aph1B and/or Aph1C gene alleles are disrupted, such that the resulting transgenic animal is homozygous for the mutation). In one embodiment of the present invention, the endogenous Aph1A and/or Aph1B and/or Aph1C gene of the transgenic animal is disrupted through homologous recombination with a nucleic acid sequence that encodes a region common to Aph1A and/or Aph1B and/or Aph1C gene products. By way of example, the disruption through homologous recombination may generate a knockout mutation in the Aph1a and/or Aph1b and/or Aph1c gene, particularly a knockout mutation wherein at least one deletion has been introduced into at least one exon of the Aph1A and/or Aph1B and/or Aph1C gene. In a preferred embodiment of the present invention, the knockout mutation is generated in a coding exon of the Aph1A and/or Aph1B and/or Aph1C gene.

Additionally a disruption in the Aph1A and/or Aph1B and/or Aph1C gene may result from insertion of a heterologous selectable marker gene into the endogenous Aph1A and/or Aph1B and/or Aph1C gene. As used herein, the term “selectable marker gene” refers to a gene encoding an enzyme that confers upon the cell or organism in which it is expressed a resistance to a drug or antibiotic, such that expression or activity of the marker can be selected for (e.g., a positive marker, such as the neo gene) or against (e.g., a negative marker, such as the dt gene). As further used herein, the term “heterologous selectable marker gene” refers to a selectable marker gene that, through experimental manipulation, has been inserted into the genome of an animal in which it would not normally be found.

The transgenic non-human animal exhibits decreased expression of functional Aph1A and/or Aph1B and/or Aph1C protein relative to a corresponding wild-type non-human animal of the same species. As used herein, the phrase “exhibits decreased expression of functional Aph1A and/or Aph1B and/or Aph1C protein” refers to a transgenic animal in whom the detected amount of functional Aph1A and/or Aph1B and/or Aph1C is less than that which is detected in a corresponding animal of the same species whose genome contains a wild-type Aph1A and/or Aph1B and/or Aph1C gene. Preferably, the transgenic animal contains at least 90% less functional Aph1A and/or Aph1B and/or Aph1C than the corresponding wild-type animal. More preferably, the transgenic animal contains no detectable, functional Aph1A and/or Aph1B and/or Aph1C as compared with the corresponding wild-type animal. Levels of Aph1A and/or Aph1B and/or Aph1C in an animal, as well as Aph1A and/or Aph1B and/or Aph1C activity, may be detected using appropriate antibodies against the Aph1A protein and/or Aph1B protein and/or Aph1C

Accordingly, where the transgenic animal of the present invention exhibits decreased expression of functional Aph1A and/or Aph1B and/or Aph1C protein relative to wild-type, the level of functional Aph1A and/or Aph1B and/or Aph1C protein in the transgenic animal is lower than that which otherwise would be found in nature. In one embodiment of the present invention, the transgenic animal expresses mutant Aph1A and/or Aph1B and/or Aph1C (regardless of amount). In another embodiment of the present invention, the transgenic animal expresses no Aph1A and/or no Aph1B and/or no Aph1C (wild-type or mutant). In yet another embodiment of the present invention, the transgenic animal expresses wild-type Aph1A and/or Aph1B and/or Aph1C protein, but at a decreased level of expression relative to a corresponding wild-type animal of the same species.

The transgenic, non-human animal of the present invention, or any transgenic, non-human animal exhibiting decreased expression of functional Aph1A and/or Aph1B and/or Aph1C protein relative to wild-type, may be produced by a variety of techniques for genetically engineering transgenic animals. For example, to create a transgenic, non-human animal exhibiting decreased expression of functional Aph1A and/or Aph1B and/or Aph1C protein relative to a corresponding wild-type animal of the same species, a Aph1A and/or Aph1B and/or Aph1C targeting vector is generated first.

As used herein, the term “Aph1A and/or Aph1B and/or Aph1C targeting vector” refers to an oligonucleotide sequence that comprises a portion, or all, of the Aph1A and/or Aph1B and/or Aph1C gene, and is sufficient to permit homologous recombination of the targeting vector into at least one allele of the endogenous Aph1A and/or Aph1B and/or Aph1C gene within the recipient cell. In one embodiment of the present invention, the targeting vector further comprises a positive or negative heterologous selectable marker gene (e.g., the positive selection gene, neo). Preferably, the targeting vector may be a replacement vector (i.e., the selectable marker gene replaces an endogenous target gene). Such a disruption is referred to herein as a “null” or “knockout” mutation. By way of example, the Aph1A and/or Aph1B and/or Aph1C targeting vector may be an oligonucleotide sequence comprising at least a portion of a non-human Aph1A and/or Aph1B and/or Aph1C gene in which there is at least one deletion in at least one exon. In a particular embodiment the Aph1A and/or Aph1B and/or Aph1C targeting vector comprises recombination sites (e.g. IoxP sites or FRT sites) which do not interrupt the coding region of the Aph1A and/or Aph1B and/or Aph1C gene.

In the method of the present invention, the Aph1A and/or Aph1B and/or Aph1C targeting vector that has been generated then may be introduced into a recipient cell (comprising a wild-type Aph1A and/or Aph1B and/or Aph1C gene) of a non-human animal, to produce a treated recipient cell. This introduction may be performed under conditions suitable for homologous recombination of the vector into at least one of the wild-type Aph1A and/or Aph1B and/or Aph1C genes in the genome of the recipient cell. The non-human animal may be any suitable animal (e.g., cat, cattle, dog, horse, goat, rodent, and sheep), as described above, but is preferably a rodent. More preferably, the non-human animal is a rat or a mouse. The recipient cell may be, for example, an embryonic stem cell, or a cell of an oocyte or zygote.

The Aph1A and/or Aph1B and/or Aph1C targeting vector of the present invention may be introduced into the recipient cell by any in vivo or ex vivo means suitable for gene transfer, including, without limitation, electroporation, DEAE Dextran transfection, calcium phosphate transfection, lipofection, monocationic liposome fusion, polycationic liposome fusion, protoplast fusion, creation of an in vivo electrical field, DNA-coated microprojectile bombardment, injection with recombinant replication-defective viruses, homologous recombination, viral vectors, and naked DNA transfer, or any combination thereof. Recombinant viral vectors suitable for gene transfer include, but are not limited to, vectors derived from the genomes of viruses such as retrovirus, HSV, adenovirus, adeno-associated virus, Semiliki Forest virus, cytomegalovirus, and vaccinia virus.

In accordance with the methods of the present invention, the treated recipient cell then may be introduced into a blastocyst of a non-human animal of the same species (e.g., by injection or microinjection into the blastocoel cavity), to produce a treated blastocyst. Thereafter, the treated blastocyst may be introduced (e.g., by transplantation) into a pseudopregnant non-human animal of the same species, for expression and subsequent germline transmission to progeny. For example, the treated blastocyst may be allowed to develop to term, thereby permitting the pseudopregnant animal to deliver progeny comprising the homologously recombined vector, wherein the progeny may exhibit decreased expression of Aph1A and/or Aph1B and/or Aph1C relative to corresponding wild-type animals of the same species. It then may be possible to identify a transgenic non-human animal whose genome comprises a disruption in its endogenous Aph1A and/or Aph1B and/or Aph1C gene. The identified transgenic animal then may be interbred with other founder transgenic animals, to produce heterozygous or homozygous non-human animals exhibiting decreased expression of functional Aph1A and/or Aph1B and/or Aph1C protein relative to corresponding wild-type animals of the same species.

A type of recipient cell for transgene introduction is the embryonal stem cell (ES). ES cells may be obtained from pre-implantation embryos cultured in vitro. Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

As used herein, a “targeted gene” or “knock-out” is a DNA sequence introduced into the germline or a non-human animal by way of human intervention, including but not limited to, the methods described herein. The targeted genes of the invention include DNA sequences which are designed to specifically alter cognate endogenous alleles.

In order to produce the gene constructs used in the invention, recombinant DNA and cloning methods, which are well known to those skilled in the art, may be utilized (see Sambrook et al., 1989, Molecular Cloning, A Laboratory Manual, 2nd Ed., Cold Spring Harbor Laboratory Press, NY). In this regard, appropriate Aph1 coding sequences may be generated from genomic clones using restriction enzyme sites that are conveniently located at the relevant positions within the Aph1 sequence. Alternatively, or in conjunction with the method above, site directed mutagenesis techniques involving, for example, either the use of vectors such as M13 or phagemids, which are capable of producing single stranded circular DNA molecules, in conjunction with synthetic oligonucleotides and specific strains of Escherichia coli (E. coli) (Kunkel, T. A. et al., 1987, Meth. Enzymol. 154:367-382) or the use of synthetic oligonucleotides and PCR (polymerase chain reaction) (Ho et al., 1989, Gene 77:51-59; Kamman, M. et al., 1989, Nucl. Acids Res. 17:5404) may be utilized to generate the necessary Aph1 (Aph1 means Aph1A and/or Aph1B and/or Aph1C) nucleotide coding sequences. Appropriate Aph1-sequences may then be isolated, cloned, and used directly to produce transgenic animals. The sequences may also be used to engineer the chimeric gene constructs that utilize regulatory sequences other than the Aph1 promoter, again using the techniques described here. These chimeric gene constructs can then also be used in the production of transgenic animals.

In a particular embodiment a non-human, transgenic animal comprising a targeting vector which further comprises recombination sites (e.g. Lox sites, FRT sites) can be crossed with a non-human, transgenic animal comprising a recombinase (e.g. Cre recombinase, FLP recombinase) under control of a particular promoter. It has been shown that these site-specific recombination systems, although of microbial origin for the majority, function in higher eukaryotes, such as plants, insects and mice. Among the site-specific recombination systems commonly used, there may be mentioned the Cre/Lox and FLP/FRT systems. The strategy normally used consists in inserting the IoxP (or FRT) sites into the chromosomes of ES cells by homologous recombination, or by conventional transgenesis, and then in delivering Cre (or FLP) for the latter to catalyze the recombination reaction. The recombination between the two IoxP (or FRT) sites may be obtained in ES cells or in fertilized eggs by transient expression of Cre or using a Cre transgenic mouse. Such a strategy of somatic mutagenesis allows a spatial control of the recombination, because the expression of the recombinase is controlled by a promoter specific for a given tissue or for a given cell. A second strategy consists in controlling the expression of recombinases over time so as to allow temporal control of somatic recombination. To do this, the expression of the recombinases is controlled by inducible promoters such as the interferon-inducible promoter, for example.

The coupling of the tetracycline-inducible expression system with the site-specific recombinase system described in WO 94 04672 has made it possible to develop a system for somatic modification of the genome which is controlled spatiotemporally. Such a system is based on the activation or repression, by tetracycline, of the promoter controlling the expression of the recombinase gene. It has been possible to envisage a new strategy following the development of chimeric recombinases selectively activated by the natural ligand for the estrogen receptor. Indeed, the observation that the activity of numerous proteins, including at least two enzymes (the tyrosine kinases c-abl and src) is controlled by estrogens, when the latter is linked to the ligand-binding domain (LBD) of the estrogen receptor alpha has made it possible to develop strategies for spatiotemporally controlled site-specific recombination. The feasibility of the site-specific somatic recombination activated by an antiestrogenic ligand has thus been demonstrated for “reporter” DNA sequences, in mice, and in particular in various transgenic mouse lines which express the fusion protein Cre-ER^{T} activated by Tamoxifen. The feasibility of the site-specific recombination activated by a ligand for a gene present in its natural chromatin environment has been demonstrated in mice.

Initial screening of the transgenic animals may be accomplished by Southern blot analysis or PCR techniques to analyze animal tissues to verify that integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and reverse transcriptase-PCR (rt-PCR). Samples of brain may be evaluated immunocytochemically using antibodies specific for Aph1A and/or Aph1B and/or Aph1C. In the present invention the transgenic mice are subjected to several behavioural and activity assays which are fully described herein in the section Materials & Methods.

In another embodiment the transgenic, non-human animal of the present invention can be used for the testing of compounds for neurodevelopmental disorders, and more specifically for the testing of compounds for neuropsychiatric disorders. Drug screening assays in general suitable for use with transgenic animals are known. See, for example U.S. Pat. Nos. 6,028,245 and 6,455,757. Thus, the transgenic animals may be used as a model system for human neurodevelopmental disorders and/or to generate neuronal cell lines that can be used as cell culture models for these disorders. The transgenic animal model systems for neurodevelopmental disorders may be used as a test substrate to identify drugs, pharmaceuticals, therapies and interventions which may be effective in treating such disorders. Therapeutic agents may be administered systemically or locally. Suitable routes may include oral, rectal, or intestinal administration; parenteral delivery, including intramuscular, subcutaneous, intramedullary injections, as well as intrathecal, intracerebral, direct intraventricular, intravenous, intraperitoneal, intranasal, or intraocular injections, just to name a few. The response of the animals to the treatment may be monitored by assessing the reversal of one or more symptoms associated with neurodevelopmental disorders. With regard to intervention, any treatments which reverse any aspect of neuronal miss-development should be considered as candidates for therapeutic intervention. However, treatments or regimes which reverse the constellation of pathologies associated with any of these disorders may be preferred. Dosages of test agents may be determined by deriving dose-response curves. The transgenic animal model systems for neuro-developmental disorders may also be used as test substrates in identifying environmental factors, drugs, pharmaceuticals, and chemicals which may exacerbate the progression of the neuropathologies that the transgenic animals exhibit. In an alternate embodiment, the transgenic animals of the invention may be used to derive a cell line which may be used as a test substrate in culture, to identify both agents that reduce and agents that enhance the neuropathologies. While primary cultures (e.g. hypocampal neurons) derived from the transgenic animals of the invention may be utilized, continuous cell lines can also be obtained. For examples of techniques which may be used to derive a continuous cell line from the transgenic animals, see Small et al., 1985, Mol. Cell. Biol. 5:642-648.

Also in another particular embodiment the transgenic non-human animal of the present invention will be useful for screening candidate therapeutic agents in order to: (1) analyze the specificity of the candidate agent; (2) monitor for side-effects of the drugs; and (3) follow long-term effects of inhibition of Aph1A and/or Aph1B and/or Aph1C activity (e.g., compensatory effects, complications, etc.).

In yet another embodiment the non-human, transgenic animal of the present invention can be used for the testing of gamma-secretase antagonists that specifically affect one of the different gamma-secretase complexes wherein said complexes lack Aph1A and/or Aph1B and/or Aph1C. In yet another embodiment cell lines derived form the non-human transgenic animals can be used for the testing of gamma-secretase antagonists that specifically affect one of the different gamma-secretase complexes wherein said complexes lack Aph1A and/or Aph1B and/or Aph1C. Thus, the present invention further provides a method for screening gamma-secretase inhibitors in transgenic animal and cells derived thereof in which an Aph1A and/or Aph1B and/or Aph1C gene is selectively inhibited. As used herein, “a gamma secretase antagonist” shall include a protein, polypeptide, peptide, nucleic acid (including DNA, RNA, and an antisense oligonucleotide), antibody (monoclonal and polyclonal, Fab fragment, F(ab′)₂ fragment) against a compound of the gamma secretase complex, molecule, compound, antibiotic, drug, and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. The antibody of the present invention may be polyclonal or monoclonal, and may be produced by techniques well known to those skilled in the art. In one embodiment of the present invention, the gamma secretase inhibitor inhibits for example cleavage of notch and/or amyloid beta precursor. In a specific embodiment only amyloid beta precursor cleavage occurs. In yet another specific embodiment gamma-secretase inhibitors can be screened (or tested) in wild type cells. Candidates of gamma-secretase inhibitors isolated via screening in wild type cells are then tested in a) cells lacking a functional expressing of Aph1A and b) in cells lacking a functional expressing of Aph1B and Aph1C. In this way candidate gamma-secretase inhibitors can be classified depending on the specificity of inhibition (for example Aph1A—specific inhibitors or combined Aph1B and Aph1C inhibitors). According to the experiments of the present invention it is expected that Aph1B and Aph1C specific inhibitors will be more suitable for the inhibition of APP processing than Aph1A specific inhibitors. However, the present invention does not exclude that Aph1A specific inhibitors are also useful for the inhibition of APP processing. Aph1A and/or Aph1B and/or Aph1C inhibitors can be used for the manufacture of medicine for the treatment of Alzheimer's disease;

It is apparent that many modifications and variations of this invention as set forth here may be made without departing from the spirit and scope thereof. The specific embodiments described below are given by way of example only and the invention is limited only by the terms of the appended claims.

### Materials and Methods

### 1. Cre-Mouse Strains

The targeted (floxed) Aph1A and/or Aph1B and/or Aph1C mice are crossed with mice where the Cre-recombinase is under control of tissue and/or organ specific promoters, under control of inducible expression or wherein the Cre-recombinase is constitutively expressed. Examples of Cre-mice used in the present invention comprise B6.Cg(SJL)-TgN(Nes-cre)1Kln, (Cre expression under control of the nestin promoter which is expressed in the central and peripheral nervous system from embryonal day E11-Jackson laboratories), B6.Cg-Tg(Syn-cre)671Jxm (Cre expression under control of the syn promoter which is expressed in neuronal cells from embryonal day E12,5—Jackson laboratories), C57BL/6J-TgN(Mx1-cre)1Cgn (inducible Cre with interferon or ds RNA—Jackson laboratories), STOCK Tg(cre/Esr1)5Amc (tamoxifen inducible Cre expression—Jackson laboratories), 129.Cg-Foxg1<tm1(cre)SKkm>(Cre expression in telencephalon—Jackson laboratories), alpha-CamKII cre (Cre expression in forebrain, Zeng et al (2001), Cell 107, 617-629), PGK-Cre (Cre expression under control of the constitutive PGK-promoter, Jackson laboratories).

### 2. Analysis of Social Behaviors

### Home Cage Behavioral Videorecording

Two pairs of cages (n=3-4 mice per cage), one of wild-type and one of mutant mice, is videorecorded simultaneously for 15 hours (10 hours during the dark cycle and 5 hours during the light cycle) for a total of 30 hours of videorecording. Various home cage behaviors are scored by two experimenters from 1 hour of the dark cycle and 1 hour of the light cycle. Whisker trimming and barbering is analyzed. Number of interactions, social grooming, mounting, tail pulling and sniffing are scored as well (Lijam et al., 1997)

### Social Dominance Tube Test

Wild-type and mutant mice are tested as previously described (Messeri et al., 1975) in a 30 cm long and 3.5 cm diameter (3.0 cm diameter for females) tube. A wild-type and a mutant mouse of the same gender are placed at opposite ends of the tube and are released. A subject is declared a “winner” when its opponent backed out of the tube. A X² one-sample analysis is used to determine if the number of wins by mutant animals is significantly different than chance.

### Nesting Patterns

Normal mice build fluffy and well-formed nests. Disturbances in these behaviors indicate altered social behavior. Six cages of wild-type and six cages of mutant mice (N=4 mice per cage) are used to evaluate nesting patterns. A 5×5 cm piece of cotton nesting material is placed in each cage. After 45 min, photographs are taken of each nest and the nest depth is measured. Nest height data are analyzed using the Student's t test.

### Home Cage Sleeping Behavior

Wild-type and mutant mice (N=4 mice per cage) are observed in their home cage, and the position and behavior of each mouse is recorded. Normal mice sleep huddled together. The percentage of subjects sleeping huddled in the same quadrant in each cage is determined. Nine observations are made over a 5-day test period. Data are analyzed by a two-way analysis of variance (ANOVA) with repeated measures.

### Whisker Trimming

The percentage of subjects having a full complement of whiskers at several ages are recorded. Data is analyzed using a X² test for independent samples. To determine if whisker loss observed in wild-type mice results from social interactions when housed with other wild-type mice, a wild-type mouse is housed with a mutant mouse. After 2, 4, and 6 weeks, the presence of whiskers in both wild-type and mutant mice is recorded, and a X² repeated 2×2 analysis is used to determine if whisker changes are significant. In the second phase, wild-type and mutant mice are returned to their original housing cage, and the presence of whiskers is recorded weekly. A separate X² repeated 2×2 analysis is used for phase 2 to determine if the change in whiskers is significant when mice are returned to their original home cage.

### Measurement of Startle and Prepulse Inhibition of Startle

Mice are tested in two SR-Lab Systems (e.g. San Diego Instruments, San Diego, Calif.) as previously described (Paylor and Crawley, 1997). Background noise level in each chamber is 70 dB.

### Acoustic Prepulse Inhibition of an Acoustic Startle Response

Two different groups of wild-type and mutant mice are tested (TEST 1 and TEST 2) for acoustic prepulse inhibition of an acoustic startle response. After a 5 min acclimation period, each subject in TEST 1 is presented 56 trials. Each session consists of seven trial types. Two startle trial types are 40 msec startle stimuli of either 100 or 115 dB. There are four different acoustic prepulse plus acoustic startle stimulus trials presented with the onset of a prepulse stimulus 100 msec before the onset of the startle stimulus. Each 20 msec prepulse stimulus (either 74 or 90 dB) is presented before both acoustic startle stimuli. Finally, there are trials where no stimulus is presented to measure baseline movement in the cylinders. The seven trial types are presented (15 sec intertrial interval) eight times in pseudorandom order. The startle response is recorded for 65 msec starting with the onset of the startle stimulus. Maximum startle amplitude is used as the dependent variable. Percent prepulse inhibition of a startle response is calculated: 100−[(startle response on acoustic prepulse and startle stimulus trials/startle response alone trials)×100]. Subjects in TEST 2 is presented 60 trials. Two startle stimuli are either 100 or 120 dB. The 20 msec prepulse stimuli are sounds of 74, 82, or 90 dB. Each prepulse stimulus is presented before both acoustic startle stimuli. There are three prepulse-only trials.

### Acoustic Prepulse Inhibition of a Tactile Startle Response

At least 3 days later, wild-type and mutant mice are tested for acoustic prepulse inhibition of a tactile startle response. One trial type is a 40 msec, 12 psi air puff. The 20 msec prepulse stimuli are 74, 78, 82, 86, or 90 dB sounds. Prepulse inhibition data is analyzed using three-way ANOVA with repeated measures. Two-way ANOVA with repeated measures are used to analyze startle data.

### 3. Assessment of Motor Function and Activity

### Rotarod

Mice are placed on a rotating drum, and latency to fall will be measured up to 60 sec. Mice that fall in less than 10 sec are given a second trial.

### Wire Hang

Wild-type and mutant mice are tested for their ability to hang from wire bars. Mice are placed on the bars and turned upside down, and latency to fall (maximum 60 sec) is measured. Mice that fell in less than 10 sec are given a second trial.

### Open-Field Activity

Exploratory locomotor activity of 11 wild-type and 11 mutant mice is measured in an open field (45×45 cm). Total horizontal activity for a 60 min period is used as a measure of open-field activity. The Student's t test is used to analyze rotarod, wire hang, and open-field data.

### Shock Threshold Analysis

Shock threshold testing is performed with ten wild-type and ten mutant mice. Each mouse is placed in a 20×20 cm chamber with a grid floor and given 1 sec foot shocks of increasing intensity (0.075 mA, 0.1 mA, 0.15 mA, 0.25 mA, 0.35 mA). Thresholds for flinching, jumping/running, and vocalization are determined.

### Morris Water Task

Wild-type and mutant mice are tested on the hidden platform version of the Morris water maze in a circular polypropylene (Nalgene) pool 105 cm in diameter. Each mouse is given 12 trials a day, in blocks of 4 trials for 4 consecutive days. The time taken to locate the escape platform (escape latency) is determined. After trials 36 and 48, each animal is given a 60 sec probe trial. During the probe test, the platform is removed and quadrant search times and platform crossings are measured. The data for the two probe trials are averaged. To estimate long-term retention of this task, mice are given a probe test 2 weeks after training. Escape latency data are analyzed with two-way ANOVA with repeated measures. Selective search data in probe trials are analyzed by individual one-way repeated ANOVA and post-hoc comparison tests. The Student's t test is used to directly compare training quadrant search time and platform crossing data between wild-type and mutant mice. Student's t tests will also be used to analyze training quadrant data from long-term retention probe trials.

### Generation of Aph1 Knock Out Mice

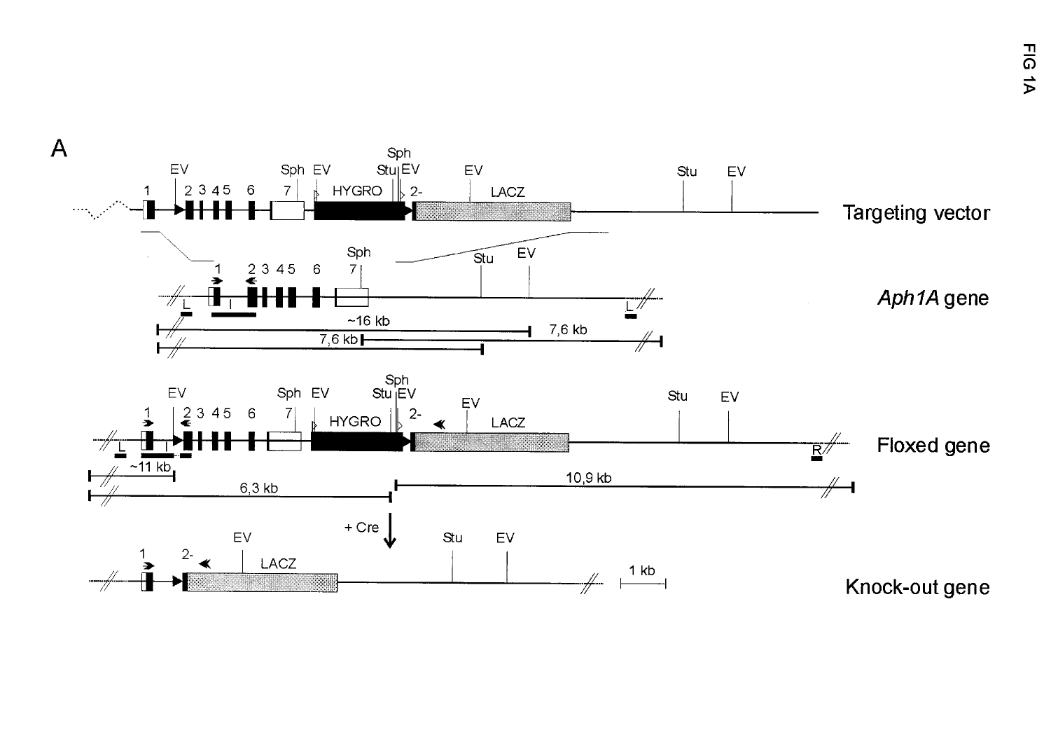

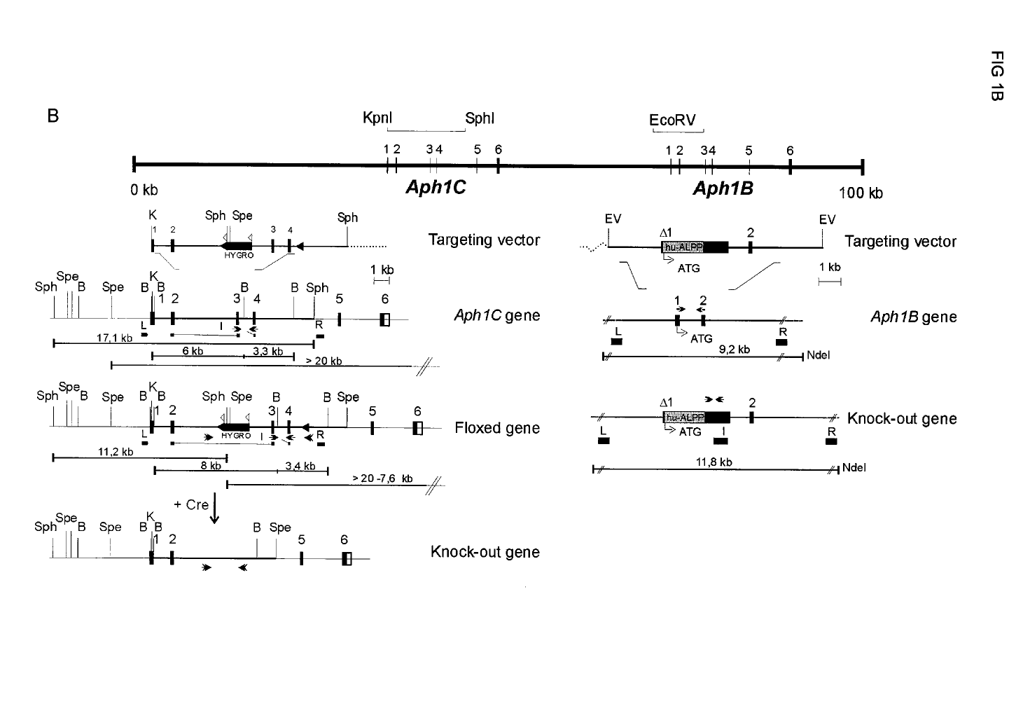

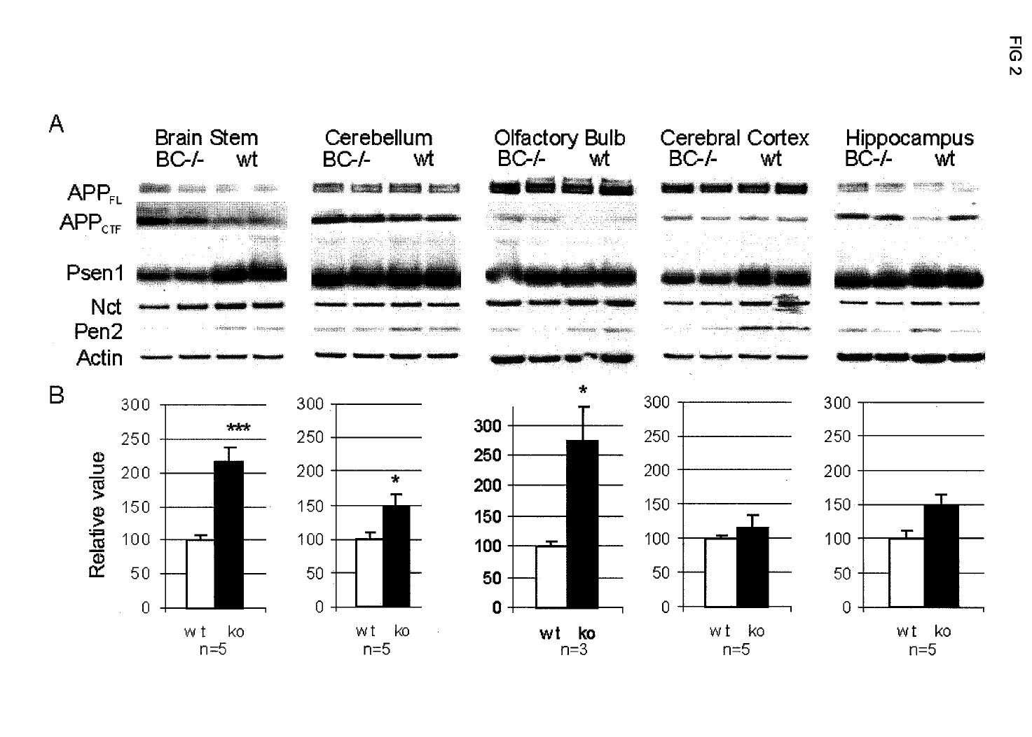

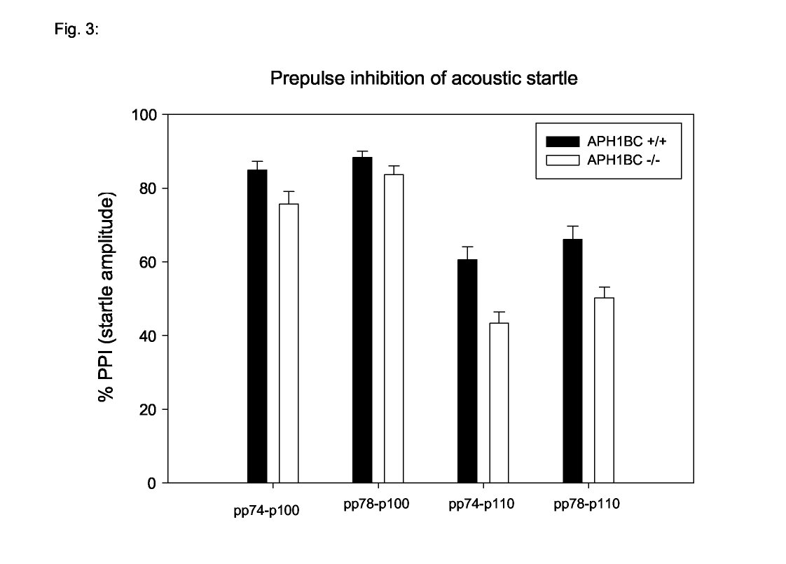

The mouse Aph1A, Aph1B and Aph1C sequences were mapped to the mouse genome using the ensemble genome browser. The mouse Aph1A gene is annotated on chromosome 3 (AC092855.39.1.249205). A pseudo-gene is linked on chromosome 1 (CAAA01207740.1.1.3729). Both Aph1C(CAAA01018252.1.1.24921) and Aph1B (CAAA01018250.1.1.45410) are linked on chromosome 9. Because Aph1C and Aph1B genes proved to be closely linked, ES cell lines are generated in which both genes are targeted on the same chromosome. A mouse cosmid clone containing the complete open reading frame of the Aph1A gene was isolated from a 129/ola cosmid library (RZPD clone id=N2362Q2). A 9.4 kb Xbal DNA restriction fragment of Aph1a covering the complete open reading frame (ATG-start codon at position 1, 7 exons and a TGA stop codon at position 2585), 536 bp 5′ sequence and 6.25 kb 3′ downstream sequence was subcloned into the plasmid vector pUC-18. The hygromycin B resistance gene, driven by the phosphoglycerate kinase (PGK) promoter flanked with two FRT sequences, one IoxP sequence downstream of the hygromycin B resistance gene, together with a LacZ reporter sequence was inserted in the Hpa I site 3′ downstream of the Aph1A gene (position 3444). The LacZ reporter sequence was constructed with a splice acceptor site at its 5′ end and a 3′ untranslated region including a polyadenylation signal. A second IoxP sequence was inserted into the Mrol site (position 540 in intron 1 (FIG. 1A). A mouse Aph1B cosmid clone containing the complete open reading frame of the Aph1B gene was isolated from a 129/ola cosmid library (RZPD clone id=M174Q2). A 6.9 kb EcoRV DNA restriction fragment of Aph1B covering 2.5 kb 5′ sequence and the first two exons (ATG-start codon at position 1) was subcloned into the plasmid vector pUC-18. Genomic sequence from Apal restriction site at position −32 (exon1) to the BamHI restriction site at position184 (intron1) was deleted. A modified human placental alkaline phosphatase (AP) reporter sequence was inserted in the Apa I site of exon1. The AP reporter sequence contains the signal peptide of CD5, a HA-tag followed by the cDNA of alkaline phosphatase (including the GPI-anchor signal sequence) and a 3′ untranslated region including a polyadenylation signal. The neomycin resistance gene driven by the thymidine kinase promoter was inserted into the BamHI site (1B). A mouse Aph1C cosmid clone containing the complete open reading frame of the Aph1C gene was isolated from a 129/ola cosmid library (RZPD clone id=F0186Q2). A 10.6 kb Knpl-Sphl DNA restriction fragment of Aph1C covering the first four exons (ATG-start codon at position 1, Kpnl site at position −6 and Sphl at position 10639) was subcloned into the plasmid vector pUC-18. The hygromycin B resistance gene, driven by the phosphoglycerate kinase (PGK) promoter flanked with two FRT sequences and one IoxP sequence upstream of the hygromycin B resistance gene was inserted in the EcoRV site (position 4554 in intron 2). A second IoxP sequence was inserted into the BgIII site (position 7085 in intron 4 (1B). The targeting vectors were linearized and introduced into the ES cell line E14 or for Aph1B into an ES cell line first targeted for Aph1C by electroporation. Hygromycin B resistant (100 μg/ml) or Neomycine resistant (200 μg/ml) colonies were screened by Southern blot analysis. Genomic DNA of Aph1A ES cells was digested with EcoRV, Stul or Sphl and hybridised either with a 5′ external gDNA probe (5′-ggaagtatgacatcaaag-3′ and 5′-tagaggttgtggggaagata-3′), internal gDNA probe (5′-gtcatgggggctgctgtgtttttc-3′ and 5′-gaaggacagagacagcagcacca-3′) or a 3′ external gDNA probe (5′-agtccatactggccctgtattca-3′ and 5′ aggcattagaatcagctcagagca-3′) as indicated in supl. FIG. 1A and displayed in 1B. Genomic DNA isolated from Aph1B ES cells was digested with Ndel and hybridised with a 5′ external gDNA probe (5′-ctgaagcctgggatgaagtt-3′ and 5′-tgtgacgtggccagtgtatt-3′), internal neomycin probe or a 3′ external gDNA probe (5′-atgcgactgttggcctatggtaaag-3′ and 5′-catatgcgtgtgtgtgtatg-3′) as indicated in supl. FIG. 1E and shown in 1F. Genomic DNA isolated from Aph1C ES cells was digested with Sphl, BamHI or Spel and hybridised either with a 5′ external gDNA probe (5′-cttgctgtggagcagctcgagga-3′ and 5′-agtggatccgaggtgactgggacg-3′), internal cDNA probe (5′-cttctggttggtgtctctcctgctt-3′ and 5′-ggagaatcaccatgaatgcccact-3′) or a 3′ external gDNA probe (5′-gctcttggctaatgcctgaagaaga-3′ and 5′ ggataacacagggttgcaacca-3′) as indicated in FIG. 1.

Mutated ES cell lines were microinjected into blastocysts of C57BL/6J mice. Chimeric males were obtained and mated with C57BL/6J females to transmit the modified Aph1 alleles to the germline. Animals carrying a null allele were obtained after breeding with transgenic females expressing a PGK driven Cre-recombinase. Determinations of the genotypes of the floxed or knock out mice or yolk sac of embryos were done by Southern blotting or PCR analysis using the probes and primers as indicated in FIG. 2. Homozygous floxed Aph-1A^{flx/flx} and Aph-1C^{flx/flx} mice were viable and fertile. Reverse transcriptase experiments on total RNA derived from the MEF's or brains of the Aph-1A^{flx/flx} and Aph1C^{flx/flx} mice demonstrated that the Aph1 mRNA was expressed from the floxed Aph1 alleles but not from the null alleles (Aph1A^{-/-}, Aph1B^{-/-} and Aph1C^{-/-}) obtained after Cre-recombinase.

### RNA Preparation and RT-PCR

Total RNA was extracted from MEF cultures grown to confluency. Briefly, cells were homogenized by scraping in Trizol® (Invitrogen), chloroform extraction was performed, RNA was precipitated by isopropanol and the RNA pellet was resuspended in deionized formamide. cDNA was generated out of 1 μg total RNA using an oligo(dT)₁₂₋₁₈-primer and SuperScript™ II Reverse-Transcriptase according to the manufacturer's instructions (Invitrogen). The following oligonucleotide primers were used to amplify cDNA's of interest: for Aph1A^{L}, 5′-TATCCAGCGCAGCCTTTCGTGCCG-3′ and 5′-CCCCCATGTTCCCTCAGTCCC-3′, for Aph1A^{S}, 5′-TATCCAGCGCAGCCTTTCGTGTAA-3′ and 5′-CAGCGAGGAGACGGAGGATGA G-3′, for simultaneous amplification of Aph1A^{L} and Aph1A^{S}, 5′-ATCACCCATCTCCATCCGACA G-3′ and 5′-GCCCAAGTGCATCAGCCAAAATA-3′. For Aph1C, 5′-TCCGCTAAGAAATCGTCCCAGTCA-3′ and 5′-CGTGAGGAGGGTGTACCACTT-3′ and for Aph1B, 5′-GACTGGCTCCCGAGGTCGT-3′ and 5′-AGGAGAGACACCAACCAG-3′.

### Histology

For immunohistochemical analysis, mice and embryos beyond E 14 were perfused via the left ventricle with either Bouin's solution diluted 1:4 in PBS or with 10% neutral buffered formaline (NBF). After dissection and overnight postfixation, individual organs were dehydrated in ascending ethanol concentrations and vacuum-embedded in low melting point paraffin (Vogel) using Clear-Rite® (Prosan) as an intermediate. The same procedure was followed with younger embryos (E 8.5-E 13.5), but here transcardiac perfusion was replaced by immersion fixation O/N on a shaker, using the same fixatives.

Serial sections (7 μm) were cut and mounted on aminosilane-coated glass slides. Central sections of each series were stained with hematoxylin and eosin for standard light microscopy. Adjacent sections were used for immunohistological screening using antibodies to glial fibrillary acid protein (astroglia), F4/80 (microglia/macrophages) and cleaved caspase 3 (apoptotic cells) For this, sections were deparaffinized in Clear-Rite (Prosan), rehydrated, sequentially blocked with hydrogen peroxide and a solution of 1% BSA plus 1% of serum from the host species in which the secondary antibody was raised. The primary antibody was applied for 1 h at RT, and after washing, was detected by a tyramide-based signal amplification technique (TSA, NEN-Dupont).

For transmission and scanning electron microscopy, Aph1A^{-/-} and wild-type embryos were fixed in 6% glutaraldehyde dissolved in Soerensen phosphate buffer (TEM) or PBS (SEM). The specimens were rinsed in the respective buffer and postfixed in 2% OsO₄ for 2 h at RT. For analysis by transmission EM, the postfixed embryos were dehydrated and then embedded in Araldite®, with propylene oxide as intermediate. The blocks were serially sectioned at 1 μm, and sections were mounted on glass slides. Every 10^{th} slide was stained by toluidine blue or p-phenylene diamine and photographed. Selected sections were re-embedded on resin stubs and re-sectioned at 70 nm for TEM. Sections were contrasted with lead citrate and photographed in a Philips CM10 transmission electron microscope.

For scanning EM, postfixed specimens after dehydration in ethanol were equilibrated with 100% acetone and dried in a Polaron CPD 7501 critical point dryer, using liquid carbon dioxide. After mounting, gold coating (‘sputtering’) was done with an AGAR automatic coater. The radioactive in situ hybridizations were preformed as reported elsewhere (6).

### Embryonic Fibroblast Culture and Recombinant Adenovirus Infection.

Mouse embryonic fibroblast cultures (MEFs) were derived form dissociated Aph1 deficient mouse embryos and their littermate controls at day 9.5 for Aph1A, at day 18.5 for Aph1C and at day 13.5 for Aph1B and Aph1AB. Outgrowing cells were subsequently immortalized by transfection with a plasmid driving expression of the large T antigen (1, 2). Cultures were maintained in DMEM/F12 containing 10% Fetal Calf serum. Replication-deficient recombinant virus AD5/dE1dE2A/CMV/NotchΔE and AD5/dE1dE2A/CMV/APP695 sw expressing NotchΔE and human APP with the Swedish mutation, respectively, were produced and purified by Galapagos Genomics ((3). Subconfluent MEF cell lines were infected with recombinant virus with a multiplicity of infection of 500. Control infections were done using a recombinant adenovirus bearing GFP cDNA at the same multiplicity of infection.

### Luciferase Reporter Cell Assays for Gamma-Secretase Cleavage

Wild-type and mutant Aph1A^{-/-} and/or Aph1B^{-/-} and/or Aph1C^{-/-} cell lines (for example mouse embryonic fibroblasts, neurons, ES cells) are generated as a tool to analyse the effects on substrate specificity caused by the absence of Aph1A and/or Aph1B and/or Aph1C. Different assays can be used. As a non-limited example a luciferase reporter assay is described. For the luciferase reporter assay wild-type and mutant Aph1A^{-/-} or Aph1B^{-/-} or Aph1C^{-/-} were plated at a density of 3×10⁴ cells in a 24 well plate and allowed to settle overnight. Each dish was transfected with 200 ng pFRluc plasmid (Stratagene) DNA and 200 ng inducer plasmid DNA APPdeltaC99-Gal4-VP16 or Gal4-VP16 using lipofectamine according to the manufacturer (Invitrogen). The cells were lysed 48 hours post transfection and luciferase activity reflecting activation of the reporter was measured with the luciferase assay system of Promega using a luminometer. All experiments were performed in triplicate. The effect linked to the gamma-secretase cleavage of substrate was determined as the ratio between the luciferase activities of the gamma-secretase dependent variant (APPdeltaC99-Gal4-VP16, NotchdeltaE-Gal4-VP16) and the mean luciferase activities of the gamma-secretase independent signal obtained with Gal4-VP16.

For the development of a cell free assay, wild-type and mutant Aph1A^{-/-} and/or Aph1BA^{-/-} and/or Aph1C^{-/-} were harvested and centrifuged. The cell pellet was resuspended in 250 mM sucrose, 5 mM Tris-HCl (pH 7,4) and 1 mM EGTA supplemented with protease inhibitors and homogenized using a ball-bearing cell cracker (10 passages, clearance 10 μm). After low-speed centrifugation (800 g, 10 minutes), the post nuclear supernatant was ultracentrifuged (100,000 g, 1 hour). The resulting microsomal pellet was washed twice in 0.02% saponin, resuspended in 5 mM Tris-1 mM EDTA (pH 7) containing 0.5% CHAPS, and incubated for 1 hr at 4° C. Next, cleared extracts (100,000 g, 1 hr) were incubated overnight (37° C.) with recombinant flag-tagged APP C100. Finally, de novo formed Aβ was analyzed by SDS-PAGE on 10% Bis-Tris NuPAGE gels (Invitrogen) in MES running buffer followed by Western blotting and ECL-detection.

It is understood that assays based on the same principles can be designed for other known gamma-secretase substrates (for example Notch, LRP, N-Cadherin, Delta, Jagged).

### Western Blot Analysis.

Cells were rinsed twice with ice-cold PBS and lysed in 1% Triton, and post-nuclear fractions were isolated by centrifugation at 10,000 g for 15 min at 4° C. Proteins were quantified using a standard Bradford assay (Pierce) and 10 μg protein/lane was loaded on Bis-Tris SDS-PAGE gels (Invitrogen) and transferred to nitrocellulose membranes for western blot detection for the indicated proteins. For Aβ intracellular detection, cells were lysed in 200 μl of ice-cold RIPA buffer (0.1% SDS, 0.5% Natrium Deoxycholate, 1% NP40, 5 mM EDTA in TBS, pH 8.0). Cleared extracts and conditioned media were used for Aβ immunoprecipitation using pAb B7/8. Immunoprecipitated samples were finally analyzed by western blotting using mAb WO2. Values were expressed as means +/− standard error of the mean (SEM).

Selected brain regions of 6 w old Aph1BC −/− mice and wt littermates were dissected and homogenized in STE-buffer. Membrane fractions were prepared by ultracentrifugation and resolubilization in 0,1M phosphate buffer (pH=5,7). Equal amounts of protein were loaded and Western blotting was performed as described.

For densitometric quantification, the films were scanned using an Image Scanner (Amersham Pharmacia) and analysed using ImageMaster™.

### Antibodies

The antibodies used for detection of Aβ were mAb WO2 (Abeta GmbH) and pAb B7/8 (4). PAbs directed against Psen1-NTF (B19.3), Psen2-CTF (B24.2), Pen-2 (B126.2), and Aph1A^{L} (B80.2) have been previously described (1, 3, 5). Antibodies against Aph1B/C were kindly provided by Dr. C. Haass (Munchen). APP was detected with pAb B63.1. mAb 9C3 recognizes the Nct C-terminus {Esselens, 2004 #1555}. Anti-myc mAb 9E10 (Sanver Tech), Anti-cleaved Notch (val 1744, Westburg), anti-N-cadherin (clone32, BD Bioscience), anti-cleaved caspase 3 (Cell Signaling Inc), anti-GFAP (Sternberger) and F4/80 protein (ATCC) were purchased.

### EXAMPLES

### 1. Aph1A Gene Targeting

To inactivate Aph1A, one IoxP sequence was introduced into intron 2, and a hygromycin resistance gene flanked by two frt sequences, one IoxP site followed by a modified beta-galactosidase was introduced downstream of aph1a. Using the 5′ external probe 9 out of 108 (8.3%) embryonic stem cell clones displayed an additional EcoRV DNA restriction fragment demonstrating homologous recombination in one of the aph1a alleles. The 9 ES clones were expanded and reanalysed with the three probes demonstrating that all ES cell lines contained a correctly targeted aph1a gene. Two ES cell clones were injected into C57BI blastocysts and resulted in coat-colour chimeric offspring. Cre-mediated excision of the region between the outermost Iox P sites in the aph1a allele generated a null allele. In this null allele a modified IacZ reporter gene (including a splice acceptor site) is located close to exon 2. If the reporter cassette is spliced onto aph1a exon2 sequence a hybrid aph1a-IacZ transcript is generated.

### 2. Aph1C Gene Targeting

To inactivate Aph1C, a hygromycin resistance gene flanked by two frt sequences and one IoxP site was introduced into intron 2. A second IoxP sequence was introduced into intron 4. Using the 5′ external probe 1 out of 72 (1.4%) embryonic stem cell clones displayed an additional SpHI DNA restriction fragment demonstrating homologous recombination in one of the aph1a alleles. This ES clone was expanded and reanalysed with the three probes demonstrating that the ES cell line contained a correctly targeted aph1b gene. This ES cell lines was injected into C57BI blastocysts and resulted in coat-colour chimeric offspring. Heterozygous knock out mice were obtained after breeding germline chimera with transgenic mice overexpressing Cre recombinase. Cre-mediated excision of the region between the two outermost IoxP sites in the aph1b gene (deletion of exon 3 and 4, deletion from AA 96 on) generated a null allele.

### 3. Aph1B Gene Targeting

To inactivate Aph1B an alkaline phosphatase (AP) reporter sequence was inserted in frame into exon1. A neomycin resistance gene was inserted in intron 2. This aph1B construct was electroporated into the ES cell line with one aph1C allele targeted. Using the 5′ external probe 2 out of 131 (1.5%) embryonic stem cell clones displayed an additional Ndel DNA restriction fragment demonstrating homologous recombination in one of the aph1B alleles. The ES clones were expanded and reanalysed with the three probes resulting in two ES cell lines with a correctly targeted aph1B gene in a cell line previously targeted for aph1C.

### 4. Analysis of Social Behaviours and Assessment of Motor Function and Physical Activity

The mutant mice are subjected to a series of social behaviour tests and motor function tests. The detailed procedures for testing are explained in the materials and methods section. With the wording “mutant mice” in it is understood a collection of the following heterozygous and/or homozygous mutant mice: (1) a general knock-out of Aph1A and/or Aph1B and/or Aph1C), (2) a knock-out of Aph1A and/or Aph1B and/or Aph1C in the central and peripheral nervous system, (3) a knock-out of Aph1A and/or Aph1B and/or Aph1C in neuronal cells, (4) a knock-out of Aph1A and/or Aph1B and/or Aph1C in the telencephalon, (5) a knock-out of Aph1A and/or Aph1B and/or Aph1C in the forebrain, (6) one or more tamoxifen induced knock-out mice generated at different time points of development, (7) one or more interferon (or dsRNA) induced knock-out generated at different time points of development.

Statistical differences are observed between the wild type and mutant mice.

### 5. The Embryonic Lethal Phenotype of Aph1A Deficient Mice is Different from the Previous Knock-Outs of γ-Secretase Components

No obvious abnormalities were observed in heterozygous Aph1A^{+/-} mice. Viable homozygous Aph1A^{-/-} mice (as defined by beating heart) were found up to embryonic day E 10.5, but never thereafter (Table 1). The first morphological indicators of abnormal development are observed after E8.5 as irregularities in the contour of the forming neural tube (neural tube “kinking” which is also observed in e.g. PS-1/2 double deficient embryos). From E 9.5 onwards, Aph1A^{-/-} mice are smaller than their wild-type littermates, and feature a moderately foreshortened body, which remains conspicuously thin caudal to the forelimb buds. In contrast to ‘full’ γ-secretase knockout phenotypes (e.g. Psen1/2^{-/-}, and Nct^{-/-}), the Aph1A^{-/-} embryos display normal embryonic turning and their caudal body axis extends further caudally at E10.5, including the regular formation of hind limb buds and a short stretch of the tail anlage. Furthermore, Aph1A^{-/-} embryos display a quite normal pattern of paraxial mesoderm segmentation something that is not observed in Notch^{-/-} or γ-secretase deficient mice. Regularly spaced, but smaller than normal somites are seen up to the level of the hind limb buds at E 10.5. The already mentioned neural tube ‘kinking’ of E 8.5 embryos is severely aggravated at E 9.5 and E 10.5. A striking abnormality of the Aph1A^{-/-} knockout mice is the failure to develop an organized vascular system in their yolk sacs. At E10.5, when wild-type yolk sacs feature a well organized vascular bed with regularly spaced 1^{st} to 3^{rd} order branches from the main vessels, only isolated blood-forming islands or short Y-shaped vascular fragments were observed. However, as nucleated blood cells are present in the vascular system of the embryo proper, we infer that a limited connection of the yolk sac vascular system to the embryo still forms.

### 6. Aph1a Deficiency Causes Apoptosis and a Novel, γ-Secretase Dependent Abnormality of Neural Tube Development

In serial semi-thin sections of E9.5 and E10.5 embryos, a novel pattern of mal-development affecting both neural tube and different mesoderm regions is identified. The characteristic strict radial orientation of the neuro-epithelial cells is disturbed, with cells aligned obliquely or even horizontally within the neural tube wall. An even more striking change is regularly seen at the outer neural tube surface, where multiple neuro-epithelial cells migrate through broad gaps in the basal lamina into the surrounding mesoderm. Furthermore groups of neural tube epithelia, surrounding mesoderm, and cranial neural crest cells undergo apoptotic cell death, as evidenced both by nuclear condensation and membrane blebbing seen in semi-thin sections as well as by an intense positive immunostaining for cleaved (activated) caspase 3. It should be noticed that in the neuronal tube also large areas existed in which no overt signs of cell death could be identified. In cross sections of the body caudal to the level of the forelimb buds many apoptotic cells in the sclerotome are observed as well. In contrast, cells within the compact dermatomyotome (which form the bulges seen by scanning EM at the surface) remain relatively preserved. Taken together, the phenotype of Aph1A^{-/-} deficient mice is quite different from other γ-secretase-deficient mouse models described in the art. Specifically, neither the neural tube migration defects nor the widespread apoptosis were described before. Also the presumably Notch/FGF8 driven segmentation in somite development is well preserved in the Aph1A^{-/-} mice compared to other γ-secretase deficient mice (that display already severe alterations after the 4^{th} or 5^{th} pair, i.e. at a level close to the forelimb bud). This striking discrepancy prompted us to re-investigate the phenotype of Psen1&2^{-/-} embryos by the same techniques applied to the Aph1A^{-/-} mice. We observed at E 9.5 cell apoptosis in mesoderm, neural crest and especially the neural tube that was even much more pronounced than in the Aph1A^{-/-} mice. Thus, in the neural tube whole groups of cells were shed into the lumen, similar to what is seen after mass apoptosis in the ventricular zone induced by for instance irradiation during cortical development. Likewise, ectopic groups of neuroepithelial cells were observed in the mesoderm surrounding the neural tube. However, misoriented neuroectoderm cells within the neural tube walls were not observed at E9.5, but were also less frequent at that stage in Aph1A−/− deficient embryos as compared to E 10.5 (a developmental stage not reached by Psen double deficient mice).

### 7. Normal Phenotype of Aph1B and Aph1C and Double Aph1Bc Deficient Mice

Both the Aph1B^{-/-} and Aph1C Aph1BC^{-/-} homozygous mice were viable and fertile, and offspring derived from heterozygous crosses were born in normal Mendelian ratio (Table 1). Microscopical inspection of tissues that express relatively high levels of Aph1B and Aph1C like brain, kidney and testis did not reveal any significant aberrations neither in routine preparations nor after detailed screening with markers for (activated) macrophages and astroglial cells.

### 8. Destabilisation of the 7-Secretase Components in Absence of Aph1A

To study the role of the different Aph1 proteins in γ-secretase complex formation we derived fibroblasts from Aph1A, Aph1B, and Aph1C deficient embryos. Microsomal membrane fractions were analysed for the expression of the different γ-secretase components. Only deficiency of Aph1A had a significant effect on Nct glycosylation, and Nct, Pen2 and Psen expression levels. Levels of Aph1B and Aph1C were not changed in the fibroblasts or in the embryo extracts. It should be noticed that the absence of Aph1B or Aph1C did also not result in increased expression levels of Aph1A protein. We next analysed the effect of the different deficiencies on γ-secretase activity in the fibroblasts by evaluating the levels of endogenous APP and N-cadherin carboxy-terminal fragments. These fragments are the direct substrates for γ-secretase and they accumulate when this activity is decreased. Again, only in Aph1A^{-/-} fibroblasts clear defects in γ-secretase processing could be demonstrated. It should be noticed that in the Aph1A^{-/-} cell lines a decreased expression of full length APP is observed as well, which could indicate a regulatory loop between APP-CTF accumulation (or inhibition of AICD generation) and APP steady state levels of expression.

### 9. APP and Notch Processing are Equally Affected by the Absence of Aph 1A

An important question is whether any of the Aph1 components differentially contributes to the cleavage of APP or Notch. Therefore we transfected fibroblasts with human APP or with an activated NotchΔE construct and measured directly the generation of Aβ peptide or NICD. Aph1A deficiency dramatically inhibited both APP and Notch processing. While Aβ generation seemed to be more strongly affected than NICD release in these experiments, it should be noticed that these assays rely on different antibodies, making it difficult to compare them directly. Therefore we transduced fibroblasts with a UAS-luciferase reporter gene and an APP or a Notch inducer construct that include a Gal4-VP16 sequence in their cytoplasmic domains. In this experiment the only variable is the inducer construct, and therefore read out can directly be compared for the two substrates. In this assay, both APP and Notch processing are affected to a similar extent by Aph-1A deficiency (about 70% inhibition). While the biochemical effect of Aph-1A deficiency (about 70% reduction in γ-secretase activity in fibroblasts) is comparable to the effect of a single Psen1 deficiency, the physiological impact of these two deficiencies on different tissues is thus quite variable. This indicates that the different γ-secretase subunit combinations fulfil specific functions in vivo. This opens the perspective of compounds that inhibit specific γ-secretase subunit combinations, which can be less toxic in the context of Alzheimer's therapy. In the context of Alzheimer therapy it is an important aim to develop inhibitors that target specifically complexes that are for instance less involved in T cell differentiation. Our experiments in the Aph1A^{-/-} mice demonstrate that this is not a purely theoretical concept. We conclude that specific subunits of the γ-secretase contribute to variable extents to specific biological functions of the complex. Aph1A for instance is very important in the yolk sac vasculogenesis, but only marginally contributing to somitogenesis.

### 10. Alterations in APP Processing in Aph1BC^{-/-} Adult Brain

Aph1BC is expressed relatively more abundantly in brain. We therefore analysed the repercussions of Aph1BC-A deficiency in different regions of adult brain on expression of the other γ-secretase subunits and APP processing (as reflected by changes in APP-CTF levels). The absence of Aph1BC affected Psen1 and Pen2 steady state levels (most clearly seen in the brain stem extracts). Aph1A expression was not significantly changed, indicating no compensatory up-regulation of this component. More importantly, in brain stem and olfactory bulb a strong, more than two-fold accumulation of APP-CTF was observed. In other brain regions a small accumulation of APP-CTF was observed that reached only statistical significance in the cerebellum (FIG. 2).

### 11. Reduced Abeta Secretion is Observed in APH1BC Deficient (Aph1BC^{-/-}) E14 Cortical Neurons

E14 embryos from APH1BC +/− crosses were dissected and cortical neurons were cultured as described in Goslin K and Banker G (1991) Culturing nerve cells, London, MIT. Single cell suspensions obtained from the cerebral cortex of individual embryos were plated on poly-L-lysine-coated plastic dishes (Nunc) in minimal essential medium (MEM) supplemented with 10% horse serum. After 4 h, culture medium was replaced by serum-free neurobasal medium with B27 supplement (GIBCO BRL). Cytosine arabinoside (5 μM) was added 24 h after plating to prevent non-neuronal (glial) cell proliferation. 72 h after plating out, recombinant SFV-huAPP₆₉₅ was diluted 10-fold in conditioned culture medium and added to the cells (1.25 ml/dish). Cultures were incubated for 1 h at 37° C., followed by incubation in conditioned medium in the absence of virus (for 2 h). Metabolic labelling was performed using methionine-free N2 medium containing 100 μCi Easy Tag Express Protein labelling mix (Perkin Elmer). After 4 h, the conditioned medium was collected and centrifuged to remove detached cells. Polyclonal B7/8, raised against the carboxyterminal 20 amino acid residues of APP ( 1/200) or Polyclonal goat antibody 207 raised against the full ectodomain of APP ( 1/200) was added to the media together with protein G-Sepharose (Pharmacia) and incubated overnight (at 4° C.). The immunoprecipitates were washed five times in DIP buffer and once in 0,3×TBS. Immunoprecipitated proteins were solubilized with NuPage™ LDS sample buffer (Invitrogen). Samples were boiled and electrophoresed on 4-12% Bis-Tris gels (Invitrogen). After fixing and drying of the gels, radiolabeled bands were detected by a Phosphorlmager (Molecular Dynamics, Inc.) and analyzed (ImageQuant 5.0). Mean Abeta secretion into the conditioned medium of APH1BC deficient and wild type littermate. E14 cortical neuronal cultures (n=4 per genotype), infected with SFV-huAPP₆₉₅. Abeta levels are normalized by sAPP_{α/β} levels to correct for SFV-infection differences. Abeta levels are significantly lowered in APH1BC deficient cultures (69% of wild-type, p=0,02).

### 12. A Prepulse Inhibition Deficit (PPI) Deficit in Aph1BC Knock-Out Mice

Schizophrenia is a complex disease characterized by delusions and hallucinations (so-called positive symptoms), affective and social disturbances (negative symptoms), but also by cognitive deficits. Disturbed information processing, and more specifically an impairment in the filtering of irrelevant stimuli, is thought to contribute to the disease phenotype by causing “sensory flooding”, which may lead to cognitive fragmentation. A psychophysical measure of (pre-attentive) information filtering is “prepulse inhibition” (PPI). When presented with a “startle stimulus”, e.g. a loud noise, humans exhibit a typical “startling” motor reaction. The strength of this reflex response is read out by recording the amplitude of the eye-blink response that is part of the startling reaction. This way, the amplitude of the eye-blink response is a measure of the efficiency of the coupling of the sensory stimulus to the motor reflex programme. If the startle stimulus is preceded by a weak, non-startling “prepulse stimulus”, e.g. a tone just above background noise levels, the amplitude of the eye-blink response to the startle stimulus is strongly diminished in normal individuals. This effect is independent of attention mechanisms, as the prepulse is presented 10-500 ms before the startle stimulus. The “% prepulse inhibition (PPI)” is quantified as: 100−((A₂/A₁)*100), with A1 being the amplitude of the response to the startle stimulus and A2 the amplitude of the response to the same startle stimulus preceded by the prepulse stimulus. This way, the % PPI is a measure of the efficiency “sensorimotor gating”: the prepulse primes the nervous system to respond less vigorously to the startle stimulus. The % PPI is decreased, and hence sensorimotor gating is less efficient, in schizophrenics, people with schizo-typical personality disorders, and to a lesser extent in blood relatives of patients with these diseases. In rodents the motor reaction to a startle stimulus is quantified by placing mice into a restraining tube in a sound-proof cabin mounted onto a pressure sensitive platform. Upon presentation of the startling sound, the mouse flinches and the pressure it exerts via its limbs is recorded quantitatively as a ballistogram that can be analysed using appropriate software. This way, the effect of a preceding prepulse on the flinching reaction to a startle stimulus can be calculated.

The APH1BC-deficient mice were put through an extensive behavioural test battery. Three month-old mice show no abnormalities in basic motor and sensory functions. They do show a significant impairment of PPI.

As can be seen in FIG. 3, different trial types were presented in a semi-random way (10 trials per type):
100 db startle stimulus alone
110 db startle stimulus alone
100 db startle stimulus preceded by a 74 db prepulse stimulus
100 db startle stimulus preceded by a 78 db prepulse stimulus
110 db startle stimulus preceded by a 74 db prepulse stimulus
110 db startle stimulus preceded by a 78 db prepulse stimulus

For all trials, background noise was 70 db, the prepulse preceded the startle stimulus by 100 ms, the prepulse stimuli lasted 20 ms and the startle stimuli lasted 60 ms. All stimuli consisted of white noise. The interval between the trials varied between 10 and 15 s. For each of the four different combinations of prepulse and startle stimulus, the % PPI was calculated using the formula described above. Compared to wild-type littermates, APH1BC-deficient mice showed a highly significantly reduced PPI for 110 db trials (p<0,001 for genotype effect in a 2-way repeated measures ANOVA with genotype and trial type as factors). For both prepulse 74/pulse 110 and prepulse 78/pulse 110 trial types, PPI in the knockouts was 70-75% of wild-type levels (post-hoc comparisons: p=0,001 for prepulse 74/pulse 110, and p=0,002 for prepulse 78/pulse 110 trials).

For 100 db trial types, there was also a PPI-impairment in the knock-outs, but it was less outspoken and only moderately significant (p=0,029 for genotype effect). Post-hoc comparisons revealed that the impairment was only significant for prepulse 74/pulse 100 trial types (p=0.011).

### Details of the Statistics:

2-way RM ANOVA voor p110 trials (factors genotype and trial type):
highly significant trial type effect (p<0,001)
highly significant genotype effect (p<0,001)
no genotype*trial type interaction
post-hoc Student-Newman-Keuls comparisons:
highly significant trial type effect within genotype groups (wt: p=0,006, ko: p=0,003)
highly significant genotype effect within trial type groups (pp 74: p=0,001, pp 78: p=0,002)
2-way RM ANOVA voor p100 trials (factors genotype and trial type):
highly significant trial type effect (p=0,002)
moderately significant genotype effect (p=0,029)
no genotype*trial type interaction
post-hoc Student-Newman-Keuls comparisons:
highly significant trial type effect only in ko group (wt: p=0,144, ko: p=0,003)
moderately significant genotype effect only in pp 74-p100 group (pp 74: p=0,011, pp 78: p=0, 190)

### 13. Effects of Anti-Psychotics on PPI in APH1BC^{-/-} Mice

A proposed common denominator of different neurodevelopmental diseases, e.g. schizophrenia and ADHD, is dysregulation of dopaminergic. This observation forms the rationale of the treatment of schizophrenia with anti-psychotics, which are all D2R-antagonists. Consistent with the hypothesis that PPI deficits in schizophrenics are indicative of an information processing deficit central in the disease etiology, antipsychotics have been shown to alleviate PPI deficits in these patients. Therefore we sought to further validate the APH1BC^{-/-} mice as a model for neurodevelopmental and especially schizophrenia-related disorders by investigating a correcting effect of antipsychotic drugs on the PPI deficit found in these mice. Haloperidol and clozapine were chosen because they are well-characterized representatives of the two major classes of antipsychotic drugs. Haloperidol is a so-called “classical” of “typical” antipsychotic, essentially limited in its action to an antagonism of D2-receptors. Clozapine is an “atypical” antipsychotic, acting upon an array of neurotransmitter receptors (e.g. different 5HT-receptors) besides its main pharmacological target, the D2-receptor. The PPI protocol was identical to the one described in the previous example. Three to six month old mice were injected successively with placebo, 1 μg/kg haloperidol or 1 μg/kg clozapine in a semi-randomized order and with sufficient time between injections (3 weeks) to avoid carry-over effects. The drugs were injected intra-parietally and PPI was measured 45 min after injection. Compared to their wild-type littermates, a highly significantly reduced PPI for 110 db trials was found in placebo-injected APH1BC^{-/-} mice, confirming the genotype effect previously found in non-injected animals (p<0,001 for genotype effect in a 2-way repeated measures ANOVA with genotype and trial type as factors, post-hoc comparisons: p=0,017 for both pp 74/p110 and pp 78/p110). Clozapine and haloperidol both essentially normalized PPI in APH1BC^{-/-} mice to wild type levels, as well in pp 74/p110 trials (p=0,139 for genotype effect in a 2-way repeated measures ANOVA with genotype and treatment regimen as factors; post-hoc comparisons: p=0,017 for genotype effect within the placebo group (see above), but p=0,791 in the CLZ group and p=0,373 in the HAL group) as in pp 78/p110 trials (p=0,237 for genotype effect in a 2-way repeated measures ANOVA with genotype and treatment regimen as factors; post-hoc comparisons: p=0,017 for genotype effect within the placebo group (see above), but p=0,608 in the CLZ group and p=0,914 in the HAL group). It should be noted that wild type PPI levels were significantly elevated in haloperidol—compared to placebo-injected animals (p=0,004 for pp74/p110, p=0,013 for pp 78/p110. No such effect was seen upon clozapine treatment. Conflicting results about the effects of different antipsychotics on PPI in wild type rodents have been reported in the literature (for review, see Geyer M A et al (2001) Psychopharmacology (Berl) 156(2-3):117-54. In the p100 trials, small but insignificant effects on (i.e. improvements of) PPI were observed after haloperidol and clozapine treatment. For these trial types, genotype differences in un-medicated animals were previously shown to be inexistent (for pp 74/p 100 trials) or very small and only moderately significant (for pp 78/p 100 trials).

### 14. Effects of Amphetamine on Locomotor Activity in Aph1BC^{-/-} Mice

Amphetamine (a dopamine agonist) use has long been known to elicit psychotic reactions in patients predisposed to schizophrenia and related diseases. When it was shown that dopaminergic signalling is dysregulated in schizophrenia, the mechanistic basis of this phenomenon became more clear, as amphetamine acts as an indirect agonist of dopamine receptors by releasing dopamine from nerve terminals. Thus, an imbalance in dopaminergic signalling may lead to a hypersensitivity to dopamine agonists. Locomotion of three to six month old APH1BC ^{-/-} mice was evaluated under illuminated conditions using an “in house made” activity monitor by measuring the number of infrared beam breaks cumulated in 5 min bins. Mice were initially placed into the activity monitor for 1 h, then injected intraparietally with placebo or 3 μg/kg amphetamine, returned to the chamber, and monitored for 2 h after injection. Placebo-injected APH1BC ^{-/-} mice do not differ significantly from their wild-type littermates in their locomotory pattern in this set-up, consistent with the results of previous tests of locomotor activity (e.g. total distance covered in an open field, 24 h activity monitoring) showing no differences between un-medicated APH1BC wt and ko mice. During the first hour of recording, locomotor activity decreased continuously as the mice habituated to their new environment. Immediately after placebo injection, there was a very transient and small activity peak, followed by a continued decline of activity during the next two hours leading to a plateau of baseline activity. For amphetamine-injected wild type as well as knock-out mice, no continued decline of activity after drug administration was seen. Instead, activity rose strongly and continuously, reaching a peak 35-40 min after injection, after which it started to decline, approximating but not quite reaching baseline levels at the end of the recording session. Interestingly and consistent with our hypothesis, APH1BC ^{-/-} mice reacted more strongly to amphetamine than their wild-type littermates, as they showed a faster rise in activity, a higher maximal activity and a higher total activity over the two hours following drug injection. The duration of the drug effect was similar for both genotypes, since activity levels became identical towards the end of the recording session. These differences were significant (p=0,06 for the genotype effect in a 2-way repeated measures ANOVA on the amphetamine-treated group with genotype and time as factors).

### 15. Effects of Apomorphine on Locomotor Activity

Hypersensitivity to apomorphine (a dopamine agonist) is another possible feature of an imbalance in dopaminergic signalling, since this drug is a direct D1/D2 receptor agonist. We examined whether apomorphine sensitivity is increased in the APH1BC ^{-/-} mice. Three to six month old mice were injected successively with placebo (1^{st} experiment) and with 2 μg/kg apomorphine (2^{nd} experiment, 3 weeks later to avoid carry-over effects). The mice were injected subcutaneously and placed into an open field apparatus for 10 min. The total distance traveled was used as a marker for locomotor activity. Placebo-injected APH1BC ^{-/-} mice do not differ significantly from their wild-type littermates in their locomotory pattern in this set-up, consistent with the results of previous tests of locomotor activity (e.g. 24 h activity monitoring) showing no differences between unmedicated APH1BC wt and ko mice, although there is a non-significant tendency for the knock-out mice to be slightly hyperactive (p=0,309). For apomorphine-injected wild type as well as knock-out mice, a strong inhibition of locomotion was seen (p<0,001 for treatment effect in a 2-way repeated measures ANOVA with genotype and treatment regimen as factors, post-hoc comparisons: p=0<0,001 for wt and ko mice). Subsequently, we expressed residual activity after apomorphine injection as total distance traveled in the open field after apomorphine injection divided by total distance traveled in the open field after placebo injection (residual activity=(distance “APO”/distance “placebo”)*100%). Interestingly APH1BC ^{-/-} mice reacted more strongly to apomorphine than their wild-type littermates; while the residual activity in wild type mice was still 50%, it was lowered to 27% in APH1BC −/− mice, and this effect was significant (p=0,045 for two-tailed student's t test).

### Tables

TABLE 1
Progenies of crosses of Aph1 heterozygous mice The value between
brackets indicates 5 additional Aph-1A^{-/-}embryos that were
recovered but probably dead as defined by no beating heart.

| | | Total | Genotype (n) | | |
|---|---|---|---|---|---|
| | Age | (n) | +/+ | +/− | −/− |
| Aph-1A | E8.5 | 43 | 13 | 20 | 10 |
| | E9.5 | 74 | 22 | 33 | 19 |
| | E10.5 | 36 | 9 | 20 | 4 (5) |
| | E11.5 | 10 | 4 | 6 | 0 |
| | 3 weeks | 166 | 63 | 103 | 0 |
| Aph-1B | 3 weeks | 67 | 21 | 32 | 14 |
| Aph-1C | 3 weeks | 248 | 75 | 109 | 64 |
| Aph-1BC | 3 weeks | 84 | 17 | 51 | 16 |

### REFERENCES

Annaert, W., and B. De Strooper. 2002. A Cell Biological Perspective on Alzheimer's Disease. Annu Rev Cell Dev Biol. 16:16.
Ballard, T. M., M. Pauly-Evers, G. A. Higgins, A. M. Ouagazzal, V. Mutel, E. Borroni, J. A. Kemp, H. Bluethmann, and J. N. Kew. 2002. Severe impairment of NMDA receptor function in mice carrying targeted point mutations in the glycine binding site results in drug-resistant nonhabituating hyperactivity. J. Neurosci. 22:6713-23.
Borrell, J., J. M. Vela, A. Arevalo-Martin, E. Molina-Holgado, and C. Guaza. 2002. Prenatal immune challenge disrupts sensorimotor gating in adult rats. Implications for the etiopathogenesis of schizophrenia. Neuropsychopharmacology. 26:204-15.
Chen, Y., R. P. Sharma, R. H. Costa, E. Costa, and D. R. Grayson. 2002. On the epigenetic regulation of the human reelin promoter. Nucleic Acids Res. 30:2930-9.
Costa, E., J. Davis, C. Pesold, P. Tueting, and A. Guidotti. 2002. The heterozygote reeler mouse as a model for the development of a new generation of antipsychotics. Curr Opin Pharmacol. 2:56-62.
De Strooper, B., P. Saftig, K. Craessaerts, H. Vanderstichele, G. Guhde, W. Annaert, K. Von Figura, and F. Van Leuven. 1998. Deficiency of presenilin-1 inhibits the normal cleavage of amyloid precursor protein [see comments]. Nature. 391:387-90.
Donoviel, D. B., A. K. Hadjantonakis, M. Ikeda, H. Zheng, P. S. Hyslop, and A. Bernstein. 1999. Mice lacking both presenilin genes exhibit early embryonic patterning defects. Genes Dev. 13:2801-10.
Edbauer, D., E. Winkler, C. Haass, and H. Steiner. 2002. Presenilin and nicastrin regulate each other and determine amyloid beta-peptide production via complex formation. Proc Natl Acad Sci USA. 99:8666-71.
Fatemi, S. H. 2001. Reelin mutations in mouse and man: from reeler mouse to schizophrenia, mood disorders, autism and lissencephaly. Mol. Psychiatry. 6:129-33.
Fatemi, S. H. 2002. The role of Reelin in pathology of autism. Mol. Psychiatry. 7:919-20.
Fatemi, S. H., J. A. Earle, and T. McMenomy. 2000. Reduction in Reelin immunoreactivity in hippocampus of subjects with schizophrenia, bipolar disorder and major depression. Mol. Psychiatry. 5:654-63, 571.
Francis, R., G. McGrath, J. Zhang, D. A. Ruddy, M. Sym, J. Apfeld, M. Nicoll, M. Maxwell, B. Hai, M. C. Ellis, A. L. Parks, W. Xu, J. Li, M. Gurney, R. L. Myers, C. S. Himes, R. Hiebsch, C. Ruble, J. S, Nye, and D. Curtis. 2002. aph-1 and pen-2 are required for Notch pathway signaling, gamma-secretase cleavage of betaAPP, and presenilin protein accumulation. Dev Cell. 3:85-97.
Gainetdinov, R. R., A. R. Mohn, and M. G. Caron. 2001. Genetic animal models: focus on schizophrenia. Trends Neurosci. 24:527-33.
Gerber, D. J., D. Hall, T. Miyakawa, S. Demars, J. A. Gogos, M. Karayiorgou, and S. Tonegawa. 2003. Evidence for association of schizophrenia with genetic variation in the 8p21.3 gene, PPP3CC, encoding the calcineurin gamma subunit. Proc Natl Acad Sci USA. 100:8993-8.
Giros, B., M. Jaber, S. R. Jones, R. M. Wightman, and M. G. Caron. 1996. Hyperlocomotion and indifference to cocaine and amphetamine in mice lacking the dopamine transporter. Nature. 379:606-12.
Goutte, C., M. Tsunozaki, V. A. Hale, and J. R. Priess. 2002. APH-1 is a multipass membrane protein essential for the Notch signaling pathway in Caenorhabditis elegans embryos. Proc Natl Acad Sci USA. 99:775-779.
Handler, M., X. Yang, and J. Shen. 2000. Presenilin-1 regulates neuronal differentiation during neurogenesis. Development. 127:2593-2606.
Hartmann, D., B. D. Strooper, and P. Saftig. 1999. Presenilin-1 deficiency leads to loss of Cajal-Retzius neurons and cortical dysplasia similar to human type 2 lissencephaly. Curr Biol. 9:719-727.
Herreman, A., D. Hartmann, W. Annaert, P. Saftig, K. Craessaerts, L. Serneels, L. Umans, V. Schrijvers, F. Checker, H. Vanderstichele, V. Baekelandt, R. Dressel, P. Cupers, D. Huylebroeck, A. Zwijsen, F. Van Leuven, and B. De Strooper. 1999. Presenilin 2 deficiency causes a mild pulmonary phenotype and no changes in amyloid precursor protein processing but enhances the embryonic lethal phenotype of presenilin 1 deficiency. Proc Natl Acad Sci USA. 96:11872-7.
Impagnatiello, F., A. R. Guidotti, C. Pesold, Y. Dwivedi, H. Caruncho, M. G. Pisu, D. P. Uzunov, N. R. Smalheiser, J. M. Davis, G. N. Pandey, G. D. Pappas, P. Tueting, R. P. Sharma, and E. Costa. 1998. A decrease of reelin expression as a putative vulnerability factor in schizophrenia. Proc Natl Acad Sci USA. 95:15718-23.
Kato, M., and W. B. Dobyns. 2003. Lissencephaly and the molecular basis of neuronal migration. Hum Mol. Genet. 12:R89-96.
Lee, S. F., S. Shah, H. Li, C. Yu, W. Han, and G. Yu. 2002. Mammalian APH-1 interacts with presenilin and nicastrin and is required for intramembrane proteolysis of amyloid-beta precursor protein and Notch. J Biol. Chem. 277:45013-9.
Li, Y. M., M. Xu, M. T. Lai, Q. Huang, J. L. Castro, J. DiMuzio-Mower, T. Harrison, C. Lellis, A. Nadin, J. G. Neduvelil, R. B. Register, M. K. Sardana, M. S. Shearman, A. L. Smith, X. P. Shi, K. C. Yin, J. A. Shafer, and S. J. Gardell. 2000. Photoactivated gamma-secretase inhibitors directed to the active site covalently label presenilin 1. Nature. 405:689-94.
Lijam, N., R. Paylor, M. P. McDonald, J. N. Crawley, C. X. Deng, K. Herrup, K. E. Stevens, G. Maccaferri, C. J. McBain, D. J. Sussman, and A. Wynshaw-Boris. 1997. Social interaction and sensorimotor gating abnormalities in mice lacking Dvl1. Cell. 90:895-905.
Messeri, P., B. E. Eleftheriou, and A. Oliverio. 1975. Dominance behavior: a phylogenetic analysis in the mouse. Physiol Behav. 14:53-8.
Miyakawa, T., L. M. Leiter, D. J. Gerber, R. R. Gainetdinov, T. D. Sotnikova, H. Zeng, M. G. Caron, and S. Tonegawa. 2003. Conditional calcineurin knockout mice exhibit multiple abnormal behaviors related to schizophrenia. Proc Natl Acad Sci USA. 100:8987-92.
Mohn, A. R., R. R. Gainetdinov, M. G. Caron, and B. H. Koller. 1999. Mice with reduced NMDA receptor expression display behaviors related to schizophrenia. Cell. 98:427-36.
Nyabi O. Bentahir M, Horre K, Herreman A, Gottardi-Littell N. Van Broeckhoven C, Merchiers P. Spittaels K, Annaert W. De Strooper B. 2003. Presenilins mutated at Asp257 or Asp385 restore Pen-2 expression and Nicastrin glycosylation but remain catalytically inactive in the absence of wild type Presenilin. J Biol. Chem. Epub ahead of print
Paylor, R., and J. N. Crawley. 1997. Inbred strain differences in prepulse inhibition of the mouse startle response. Psychopharmacology (Berl). 132:169-80.
Shen, J., R. T. Bronson, D. F. Chen, W. Xia, D. J. Selkoe, and S. Tonegawa. 1997. Skeletal and CNS defects in Presenilin-1-deficient mice. Cell. 89:629-39.
Sherrington, R., E. I. Rogaev, Y. Liang, E. A. Rogaeva, G. Levesque, M. Ikeda, H. Chi, C. Lin, G. Li, K. Holman, and et al. 1995. Cloning of a gene bearing missense mutations in early-onset familial Alzheimer's disease [see comments]. Nature. 375:754-60.
Shi, L., S. H. Fatemi, R. W. Sidwell, and P. H. Patterson. 2003. Maternal influenza infection causes marked behavioral and pharmacological changes in the offspring. J. Neurosci. 23:297-302.
Struhl, G., and A. Adachi. 2000. Requirements for presenilin-dependent cleavage of notch and other transmembrane proteins [In Process Citation]. Mol. Cell. 6:625-36.
Thinakaran, D. R. Borchelt, M. K. Lee, H. H. Slunt, L. Spitzer, G. Kim, T. Ratovitsky, F. Davenport, C. Nordstedt, M. Seeger, J. Hardy, A. I. Levy, S. E. Gandy, N. A. Jenkins, N. G. Copeland, D. L. Price, and S. Sisodia. 1996. Endoproteolysis of presenilin 1 and accumulation of processed derivatives in vivo. Neuron. 17:181-190.
Tueting, P., E. Costa, Y. Dwivedi, A. Guidotti, F. Impagnatiello, R. Manev, and C. Pesold. 1999. The phenotypic characteristics of heterozygous reeler mouse. Neuroreport. 10:1329-34.
Wolfe, M. S., W. Xia, B. L. Ostaszewski, T. S. Diehl, W. T. Kimberly, and D. J. Selkoe. 1999. Two transmembrane aspartates in presenilin-1 required for presenilin endoproteolysis and gamma-secretase activity. Nature. 398:513-517.
Wood, G. K., H. Tomasiewicz, U. Rutishauser, T. Magnuson, R. Quirion, J. Rochford, and L. K. Srivastava. 1998. NCAM-180 knockout mice display increased lateral ventricle size and reduced prepulse inhibition of startle. Neuroreport. 9:461-6.
Yu, G., M. Nishimura, S. Arawaka, D. Levitan, L. Zhang, A. Tandon, Y. Q. Song, E. Rogaeva, F. Chen, T. Kawarai, A. Supala, L. Levesque, H. Yu, D. S. Yang, E. Holmes, P. Milman, Y. Liang, D. M. Zhang, D. H. Xu, C. Sato, E. Rogaev, M. Smith, C. Janus, Y. Zhang, R. Aebersold, L. S. Farrer, S. Sorbi, A. Bruni, P. Fraser, and P. St George-Hyslop. 2000. Nicastrin modulates presenilin-mediated notch/glp-1 signal transduction and betaAPP processing. Nature. 407:48-54.

1. A transgenic, non-human animal characterised by having an endogenous nucleic acid sequence encoding a non-functional aph1a and/or aph1b and/or aph1c.

2. A transgenic, non-human animal according to claim 1 wherein said non-functional aph1a and/or aph1b and/or aph1c expression is in a specific tissue or in a specific organ.

3. A transgenic, non-human animal according to claim 1 wherein said non-functional expression of aph1a and/or aph1b and/or aph1c results in a neurodevelopmental disorder that displays symptoms relevant for schizophrenia and/or bipolar disorder and/or depression and/or a compulsive disorder and/or lissencephaly and/or autism and/or an attention deficit hyperactivity disorder and/or mental retardation.

4. A transgenic, non-human animal according to claim 1 wherein said animal is a rodent.

5. Cell lines derived from the transgenic animals according to claim 1.

6. Cell lines according to claim 5 wherein said cells are primary neurons.

7. An isolated gamma-secretase complex lacking aph1a and/or aph1b and/or aph1c.

8. Use of a transgenic animal according to claim 1 for screening compounds capable of preventing or treating neurodevelopmental disorders.

9. Use of a transgenic animal according to claim 1 for testing gamma-secretase antagonists that specifically modulate gamma-secretase complexes lacking aph1a and/or aph1b and/or aph1c.

10. Use of cell lines according to claim 5 for screening gamma-secretase inhibitors and/or for testing candidate gamma-secretase inhibitors.
